SEQUENCE LISTING (11 sequences)

SEQ ID NO: 1 - DNA, 1109 bp, Homo sapiens
  source 1..1109: mol_type = genomic DNA
  CDS 118..999
gcttcgccggggccgggcggccggcgcccccggctgctcccgccgccgcccggacccgcgccccgccggggcagcggtggtgagagccccgactccccggacgccgcccgccgtgccatggggttcccggccgcggcgctgctctgcgcgctgtgctgcggcctcctggccccggctgcccgcgccggctactccgaggagcgctgcagctggaggggcagcggcctcacccaggagcccggcagcgtggggcagctggccctggcctgtgcggagggcgcggttgagtggctgtacccggctggggcgctgcgcctgaccctgggcggccccgatcccagagcgcggcccggcatcgcctgtctgcggccggtgcggcccttcgcgggcgcccaggtcttcgcggagcgcgcagggggcgccctggagctgctgctggccgagggcccgggcccggcagggggccgctgcgtgcgctggggtccccgcgagcgccgggccctcttcctgcaggccacgccgcaccaggacatcagccgccgcgtggccgccttccgctttgagctgcgcgaggacgggcgccccgagctgcccccgcaggcccacggtctcggcgtagacggtgcctgcaggccctgcagcgacgctgagctgctcctggccgcatgcaccagcgacttcgtaattcacgggatcatccatggggtcacccatgacgtggagctgcaggagtctgtcatcactgtggtggccgcccgtgtcctccgccagacaccgccgctgttccaggcggggcgatccggggaccaggggctgacctccattcgtaccccactgcgctgtggcgtccacccgggcccaggcaccttcctcttcatgggctggagccgctttggggaggcccggctgggctgtgccccacgattccaggagttccgccgtgcctacgaggctgcccgtgctgcccacctccacccctgcgaggtggcgctgcactgaggggctgggtgctggggaggggctggtaggagggagggtgggcccactgctttggaggtgatgggactatcaataagaactctgttcacgcaaaaaaaaaaaaaaaaaaa

SEQ ID NO: 2 - protein, 293 aa, Homo sapiens
MGFPAAALLCALCCGLLAPAARAGYSEERCSWRGSGLTQEPGSVGQLALACAEGAVEWLYPAGALRLTLGGPDPRARPGIACLRPVRPFAGAQVFAERAGGALELLLAEGPGPAGGRCVRWGPRERRALFLQATPHQDISRRVAAFRFELREDGRPELPPQAHGLGVDGACRPCSDAELLLAACTSDFVIHGIIHGVTHDVELQESVITVVAARVLRQTPPLFQAGRSGDQGLTSIRTPLRCGVHPGPGTFLFMGWSRFGEARLGCAPRFQEFRRAYEAARAAHLHPCEVALH

SEQ ID NO: 3 - protein, 270 aa, Homo sapiens
GYSEERCSWRGSGLTQEPGSVGQLALACAEGAVEWLYPAGALRLTLGGPDPRARPGIACLRPVRPFAGAQVFAERAGGALELLLAEGPGPAGGRCVRWGPRERRALFLQATPHQDISRRVAAFRFELREDGRPELPPQAHGLGVDGACRPCSDAELLLAACTSDFVIHGIIHGVTHDVELQESVITVVAARVLRQTPPLFQAGRSGDQGLTSIRTPLRCGVHPGPGTFLFMGWSRFGEARLGCAPRFQEFRRAYEAARAAHLHPCEVALH

SEQ ID NO: 4 - DNA, 1363 bp, Mus musculus
  source 1..1363: mol_type = genomic DNA
  CDS 84..959
gggcagccgcgccgcgggctgctcgcgctgcggccccgaccctcccggggcagcagtccgaggccccggcgcgtcccctaaccatgctggtagccacgcttctttgcgcgctctgttgcggcctcctggccgcgtccgctcacgctggctactcggaagaccgctgcagctggaggggcagcggtttgacccaggagcctggcagcgtggggcagctgaccctggactgtactgagggcgctatcgagtggctgtacccagctggggcgctgcgcctgaccctgggcggccccgatccgggcacacggcccagcatcgtctgtctgcgcccagagcggcccttcgctggtgcccaggtcttcgctgaacgtatgaccggcaatctagagttgctactggccgagggcccggacctggctgggggccgctgcatgcgctggggtccccgcgagcgccgagcccttttcctgcaggccacaccacaccgcgacatcagccgcagagttgctgccttccgttttgaactgcacgaggaccaacgtgcagaaatgtctccccaggctcaaggtcttggtgtggatggtgcctgcaggccctgcagtgatgccgagctcctcctggctgcatgcaccagtgattttgtgatccacgggaccatccatggggtcgcccatgacacagagctgcaagaatcagtcatcactgtggtggttgctcgtgtcatccgccagacactgccactgttcaaggaagggagctcggagggccaaggccgggcctccattcgtaccttgctgcgctgtggtgtgcgtcctggcccaggctccttcctcttcatgggctggagccgatttggcgaagcttggctgggctgtgctccccgcttccaagagttcagccgtgtctattcagctgctctcacgacccatctcaacccatgtgagatggcactggactgagagacctgggagcaagccctggatggaccttcttctggagatggggtgttggggagggtgatgggagggtgggtgagaagggtgtggctcggatggcatcctggtacccacagtgagctggtagaatactaagtaatctggaccataccagccactgtagtcatggtcttctgtggcaggcagcatacccagctctgtgcctgcctcactttgtctactctccagtctgctgcccttctaacccttcttagcctgctgaccagtgagctcatgttttcctcgaattccagggtgctgctggggttcagagcaaccgtgccgtagtttggaagacttgagctaattgttttttttttgtttgtttttttgtttgtttaaaggtggcctggggggggcggcaaaca

SEQ ID NO: 5 - protein, 291 aa, Mus musculus
MLVATLLCALCCGLLAASAHAGYSEDRCSWRGSGLTQEPGSVGQLTLDCTEGAIEWLYPAGALRLTLGGPDPGTRPSIVCLRPERPFAGAQVFAERMTGNLELLLAEGPDLAGGRCMRWGPRERRALFLQATPHRDISRRVAAFRFELHEDQRAEMSPQAQGLGVDGACRPCSDAELLLAACTSDFVIHGTIHGVAHDTELQESVITVVVARVIRQTLPLFKEGSSEGQGRASIRTLLRCGVRPGPGSFLFMGWSRFGEAWLGCAPRFQEFSRVYSAALTTHLNPCEMALD

SEQ ID NO: 6 - protein, 270 aa, Mus musculus
GYSEDRCSWRGSGLTQEPGSVGQLTLDCTEGAIEWLYPAGALRLTLGGPDPGTRPSIVCLRPERPFAGAQVFAERMTGNLELLLAEGPDLAGGRCMRWGPRERRALFLQATPHRDISRRVAAFRFELHEDQRAEMSPQAQGLGVDGACRPCSDAELLLAACTSDFVIHGTIHGVAHDTELQESVITVVVARVIRQTLPLFKEGSSEGQGRASIRTLLRCGVRPGPGSFLFMGWSRFGEAWLGCAPRFQEFSRVYSAALTTHLNPCEMALD

SEQ ID NO: 7 - DNA, 1026 bp, Rattus norvegicus
  source 1..1026: mol_type = genomic DNA
  CDS 1..876
atgctggtagcggcgcttctctgcgcgctgtgctgcggcctcttggctgcgtccgctcgagctggctactccgaggaccgctgcagctggaggggcagcggtttgacccaggaacctggcagcgtggggcagctgaccctggattgtactgagggtgctatcgagtggctgtatccagctggggcgctgcgcctgactctaggcggctctgatccgggcacgcggcccagcatcgtctgtctgcgcccaacacggcccttcgctggtgcccaggtcttcgctgaacggatggccggcaacctagagttgctactggccgagggccaaggcctggctgggggccgctgcatgcgctggggtcctcgcgagcgccgagcccttttcctgcaggccacgccacaccgggacatcagccgcagagttgctgccttccaatttgaactgcacgaggaccaacgtgcagaaatgtctccccaggcccaaggttttggtgtggatggtgcctgcaggccctgcagtgatgccgagctccttctgactgcatgcaccagtgactttgtgatccatgggaccatccatggggtcgtccatgacatggagctgcaagaatcagtcatcactgtggtggccactcgtgtcatccgccagacactgccactgttccaggaagggagctcggagggccggggccaggcctccgttcgtaccttgttgcgctgtggtgtgcgtcctggcccaggctccttcctcttcatgggctggagccgatttggcgaagcttggctgggctgcgctccccgcttccaagagttcagccgtgtctattcagctgctctcgcggcccacctcaacccatgtgaggtggcactggactgagagacctgggagcaagccctggatggatcttcctctggggatggggtgttggggaggggtgataggagggtgggtgggaagggtgtggctcagatggcatcctggtacccacagtgaggtggtagaatactaaataacctggatcacacc

SEQ ID NO: 8 - protein, 291 aa, Rattus norvegicus
MLVAALLCALCCGLLAASARAGYSEDRCSWRGSGLTQEPGSVGQLTLDCTEGAIEWLYPAGALRLTLGGSDPGTRPSIVCLRPTRPFAGAQVFAERMAGNLELLLAEGQGLAGGRCMRWGPRERRALFLQATPHRDISRRVAAFQFELHEDQRAEMSPQAQGFGVDGACRPCSDAELLLTACTSDFVIHGTIHGVVHDMELQESVITVVATRVIRQTLPLFQEGSSEGRGQASVRTLLRCGVRPGPGSFLFMGWSRFGEAWLGCAPRFQEFSRVYSAALAAHLNPCEVALD

SEQ ID NO: 9 - protein, 270 aa, Rattus norvegicus
GYSEDRCSWRGSGLTQEPGSVGQLTLDCTEGAIEWLYPAGALRLTLGGSDPGTRPSIVCLRPTRPFAGAQVFAERMAGNLELLLAEGQGLAGGRCMRWGPRERRALFLQATPHRDISRRVAAFQFELHEDQRAEMSPQAQGFGVDGACRPCSDAELLLTACTSDFVIHGTIHGVVHDMELQESVITVVATRVIRQTLPLFQEGSSEGRGQASVRTLLRCGVRPGPGSFLFMGWSRFGEAWLGCAPRFQEFSRVYSAALAAHLNPCEVALD

SEQ ID NO: 10 - DNA, 882 bp, synthetic construct
  misc_feature 1..882: note = Codon optimised
  source 1..882: mol_type = other DNA
atgggctttcccgctgccgccctgctgtgcgctctgtgctgcggactgctggctcctgcagccagagccggctacagcgaggaacggtgcagctggcggggcagcggcctgacccaggaacctggcagcgtcggccagctcgcactggcctgtgcagaaggcgccgtggagtggctgtaccccgcaggcgccctgagactgaccctgggcggacccgaccccagagccagacccggcattgcctgtctgaggcccgtgcggcctttcgctggcgcccaggtgttcgccgagagagccggcggagccctggaactcctgctcgccgaaggccctggtccagccggcggaagatgcgtgagatggggcccaagagagcggagagccctgttcctgcaagccaccccccaccaggacatcagcagacgggtggccgccttcagattcgagctgcgggaggacggtagacccgagctgccacctcaggcccacggactgggagtggacggcgcctgcagaccctgtagcgacgccgagctgctgctcgccgcctgcaccagcgacttcgtgatccacggcatcatccacggcgtgacccacgacgtggagctgcaggaaagcgtcatcaccgtcgtcgccgccagagtgctgagacagaccccccctctgttccaggccggcagaagcggcgaccagggcctgaccagcatccggacccccctgagatgcggcgtgcatcccggacccggcaccttcctgttcatgggctggtccagattcggcgaggcccggctgggctgcgctccccggttccaggaattcagacgggcctacgaggccgccagggccgctcatctgcacccctgcgaggtggccctgcattga

SEQ ID NO: 11 - protein, 270 aa, synthetic construct
  VARIANT 1..270: note = Consensus sequence
  VARIANT 5: note = misc_feature - Xaa can be any naturally occurring amino acid
  VARIANT 25: note = misc_feature - Xaa can be any naturally occurring amino acid
  VARIANT 27: note = misc_feature - Xaa can be any naturally occurring amino acid
  VARIANT 29: note = misc_feature - Xaa can be any naturally occurring amino acid
  VARIANT 33: note = misc_feature - Xaa can be any naturally occurring amino acid
  VARIANT 49: note = misc_feature - Xaa can be any naturally occurring amino acid
  VARIANT 52..53: note = misc_feature - Xaa can be any naturally occurring amino acid
  VARIANT 56: note = misc_feature - Xaa can be any naturally occurring amino acid
  VARIANT 58: note = misc_feature - Xaa can be any naturally occurring amino acid
  VARIANT 63: note = misc_feature - Xaa can be any naturally occurring amino acid
  VARIANT 76..77: note = misc_feature - Xaa can be any naturally occurring amino acid
  VARIANT 79: note = misc_feature - Xaa can be any naturally occurring amino acid
  VARIANT 88..90: note = misc_feature - Xaa can be any naturally occurring amino acid
  VARIANT 96: note = misc_feature - Xaa can be any naturally occurring amino acid
  VARIANT 114: note = misc_feature - Xaa can be any naturally occurring amino acid
  VARIANT 124: note = misc_feature - Xaa can be any naturally occurring amino acid
  VARIANT 128: note = misc_feature - Xaa can be any naturally occurring amino acid
  VARIANT 131: note = misc_feature - Xaa can be any naturally occurring amino acid
  VARIANT 133: note = misc_feature - Xaa can be any naturally occurring amino acid
  VARIANT 135..136: note = misc_feature - Xaa can be any naturally occurring amino acid
  VARIANT 140: note = misc_feature - Xaa can be any naturally occurring amino acid
  VARIANT 142: note = misc_feature - Xaa can be any naturally occurring amino acid
  VARIANT 159: note = misc_feature - Xaa can be any naturally occurring amino acid
  VARIANT 170: note = misc_feature - Xaa can be any naturally occurring amino acid
  VARIANT 175: note = misc_feature - Xaa can be any naturally occurring amino acid
  VARIANT 178: note = misc_feature - Xaa can be any naturally occurring amino acid
  VARIANT 189..190: note = misc_feature - Xaa can be any naturally occurring amino acid
  VARIANT 193: note = misc_feature - Xaa can be any naturally occurring amino acid
  VARIANT 197: note = misc_feature - Xaa can be any naturally occurring amino acid
  VARIANT 201..202: note = misc_feature - Xaa can be any naturally occurring amino acid
  VARIANT 204: note = misc_feature - Xaa can be any naturally occurring amino acid
  VARIANT 206..208: note = misc_feature - Xaa can be any naturally occurring amino acid
  VARIANT 210..211: note = misc_feature - Xaa can be any naturally occurring amino acid
  VARIANT 213: note = misc_feature - Xaa can be any naturally occurring amino acid
  VARIANT 216: note = misc_feature - Xaa can be any naturally occurring amino acid
  VARIANT 222: note = misc_feature - Xaa can be any naturally occurring amino acid
  VARIANT 227: note = misc_feature - Xaa can be any naturally occurring amino acid
  VARIANT 240: note = misc_feature - Xaa can be any naturally occurring amino acid
  VARIANT 251: note = misc_feature - Xaa can be any naturally occurring amino acid
  VARIANT 253: note = misc_feature - Xaa can be any naturally occurring amino acid
  VARIANT 255: note = misc_feature - Xaa can be any naturally occurring amino acid
  VARIANT 258..260: note = misc_feature - Xaa can be any naturally occurring amino acid
  VARIANT 263: note = misc_feature - Xaa can be any naturally occurring amino acid
  VARIANT 267: note = misc_feature - Xaa can be any naturally occurring amino acid
  VARIANT 270: note = misc_feature - Xaa can be any naturally occurring amino acid
GYSEXRCSWRGSGLTQEPGSVGQLXLXCXEGAXEWLYPAGALRLTLGGXDPXXRPXIXCLRPXRPFAGAQVFAERXXGXLELLLAEGXXXAGGRCXRWGPRERRALFLQATPHXDISRRVAAFXFELXEDXRXEXXPQAXGXGVDGACRPCSDAELLLXACTSDFVIHGXIHGVXHDXELQESVITVVXXRVXRQTXPLFXXGXSXXXGXXSXRTXLRCGVXPGPGXFLFMGWSRFGEAXLGCAPRFQEFXRXYXAAXXXHLXPCEXALX

(19) United States
(12) Patent Application Publication
(10) Pub. No.: US 2025/0325627 A1
(43) Pub. Date: Oct. 23, 2025

(54) TREATMENT OF NOCICEPTIVE PAIN

(71) Applicant: Hoba Therapeutics ApS, København N (DK)

(72) Inventors: Kenneth PETERSEN, Frederiksberg (DK); Gordon MUNRO, Skovlunde (DK); Torsten MELDGAARD MADSEN, København Ø (DK)

(21) Appl. No.: 18/717,758

(22) PCT Filed: Dec. 8, 2022
(86) PCT No.: PCT/EP2022/084971

§ 371 Date: Jun. 7, 2024

(30) Foreign Application Priority Data
Dec. 10, 2021 (EP) 21213798.8

(51) Int. Cl. A61K 38/17; A61P 29/00

(52) U.S. Cl. CPC A61K 38/1709; A61P 29/00

(57) ABSTRACT

The present disclosure relates to Meteorin and its use in prevention and/or treatment of nociceptive pain. Acute and chronic nociceptive pain caused by e.g. injuries to the body or inflammation may be treated by administration of Meteorin to the patient.

### CROSS-REFERENCE TO RELATED APPLICATIONS

This application is the National Stage Application of International Patent Application No. PCT/EP2022/084971, filed on Dec. 8, 2022, which claims the benefit of European Patent Application No. 21213798.8, filed on Dec. 10, 2021, the disclosures of each of which are herein incorporated by reference in their entireties.

### SEQUENCE LISTING

The instant application contains a Sequence Listing XML which has been submitted electronically as an XML formatted sequence listing and is hereby incorporated by reference in its entirety. Said XML formatted sequence listing, created on Dec. 3, 2024, is named P5973US00_Substitute_sequence_listing.xml and is 36,810 bytes in size.

### TECHNICAL FIELD

The present invention relates to Meteorin and its use in treatment and/or prevention of nociceptive pain.

### BACKGROUND

Acute pain is an unpleasant, dynamic psychophysiological process that typically occurs in response to tissue trauma and related inflammatory processes and plays an essential role in wound healing. However, pain that persists beyond a healing period of 3 months (according to International Classification of Diseases, 11th edition criteria) serves no obvious biological purpose and is regarded as chronic in nature. The International Association for the Study of Pain which defines pain as “an unpleasant sensory and emotional experience associated with, or resembling that associated with, actual or potential tissue damage” has classified chronic pain into the three main categories: nociceptive, neuropathic and nociplastic. Nociceptive pain is the most common form of chronic pain, encompassing arthritis and most forms of spinal pain. However, there is growing recognition that many pain conditions, especially those involving cancer and spine pain, have a mixed pain phenotype. For the purpose of this application these three chronic pain categories are briefly compared to help provide precise definitions.

### Nociceptive Pain Versus Neuropathic and Nociplastic Pains

Nociceptive pain results from tissue or potential tissue damage. Typical examples include degenerative changes that occur via normal wear and tear (degenerative disc disease, facet arthropathy, primary osteoarthritis), trauma, (eg, burns, muscle tears, traumatic arthritis), muscle spasm, visceral pathology (eg, ulcers, renal stones, pancreatitis). It is typically described as having a throbbing or aching quality, and in contrast to neuropathic pain is rarely associated with sensory deficits (eg, numbness, tingling, pricking). Nociceptive hypersensitivity is generally restricted to the immediate area of injury which again contrasts with neuropathic pain which is commonly associated with non-painful stimuli (allodynia) and radiates distally in a nerve or nerve root. Nociceptive pain can be treated successfully with opioid analgesics, which are increasingly avoided due to safety and tolerability issues, non-steroidal anti-inflammatory drugs (topical and systemic), muscle relaxants (more effective for acute and subacute spinal pain), and disease modifying anti-rheumatic drugs (inflammatory arthritis).

Neuropathic pain also has an identifiable basis occurring as a consequence of disease or injury that affects the nervous system. Compared with nociceptive pain, neuropathic pain is typically associated with sensory abnormalities, such as numbness and allodynia, more prominent pain paroxysms and, depending on the nerve(s) affected, neurological findings. Neuropathic pain is generally described as having lancinating and/or shooting features. As opposed to many forms of nociceptive pain and acute nerve injury, chronic neuropathic pain is always maladaptive.

The differences between nociceptive pain and neuropathic pain are also reflected in the official treatment guidelines, as there is very little overlap between the drugs that are recommended and used to treat neuropathic pain and drugs that are recommended and used to treat nociceptive pain as detailed below.

For the pharmacologic treatment of neuropathic pain, clinical practice guidelines have been published by the International Association for the Study of Pain (Finnerup et al. 2015), the European Federation of Neurological Societies (EFNS) (Attal et al. 2010), the National Institute for Health and Care Excellence (NICE) of the UK (NICE 2013) and the Canadian Pain Society (CPS) (Moulin et al. 2014). Three drug classes have received recommendations for first-line therapy in all guidelines:
1. Tricyclic antidepressants, particularly amitriptyline;
2. The serotonin-norepinephrine reuptake inhibitors (SNRIs), such as duloxetine; and
3. The Ca²⁺ channel alpha-2-delta ligands gabapentin and pregabalin.

Tramadol, a mixed opioid/SNRI, is recommended for second-line treatment of neuropathic pain. Drugs recommended for third- and fourth-line treatment commonly include strong opioids and anti-epileptic agents other than gabapentinoids (e.g. lamotrigine), and cannabinoids.

For pharmacological treatment for nociceptive pain the WHO recommends using a three-step ladder approach:
i) For step 1, non-opioid analgesics (e.g. paracetamol) and NSAIDs (e.g. aspirin, diclofenac, ibuprofen) are recommended.
ii) For step 2, weak opioids (e.g. codeine, tramadol) can be introduced in combination with step 1 analgesics.
iii) For step 3, strong opioids, primarily morphine, can be used in combination with step 1 analgesics.

Furthermore, the differences between nociceptive pain and neuropathic pain are also reflected in the official clinical development guidelines on pain therapeutics available from EMA (EMA/CHMP/970057/2011) and FDA (FDA 34355740dft.docx Feb. 7, 2022, draft version).

Throughout the guidelines, the regulatory authorities clearly differentiate between nociceptive and neuropathic pain. It is emphasized that a pain compound intended for nociceptive pain should target the underlying etiology and disease mechanism, and the study population should be homogenous and selected by diagnosis, intensity, and duration (acute vs. chronic). A successful development will lead to approval only in the nociceptive pain indication and etiology investigated. The same applies to neuropathic pain, i.e. if a neuropathic pain drug is developed for diabetic neuropathic pain, then only this indication will be included in the label.

In the instance of mixed pain (nociplastic pain (revised IASP terminology)), a potential candidate should be investigated and be confirmed efficient in at least two different clinical programs; one in nociceptive pain and one in neuropathic pain.

In conclusion, according to the regulatory authorities, an approval of a drug candidate in one pain indication would not automatically be approved or even considered for other pain types. This acknowledges that one cannot predict whether a drug developed for neuropathic pain can or will be efficient in treating nociceptive pain and vice versa.

The third type of chronic pain termed nociplastic pain, is a type of pain that arises from the abnormal processing of pain signals without any clear evidence of tissue damage or discrete pathology involving the somatosensory system. Previously known as functional pain syndromes, these conditions include pain states such as fibromyalgia, irritable bowel syndrome, and possibly non-specific back pain. The pathophysiological mechanisms that cause these disorders primarily involve augmented sensory processing throughout the nociceptive axis and diminished functioning of inhibitory pathways within the central nervous system.

The pain therapies currently used, have only modest efficacy in most patients and their side effects represent significant limitations for their use. Hence, there is a high need of safe and effective therapies for prevention and treatment of nociceptive pain, that do not produce analgesic tolerance and have none or only minor side effects that do affect the general health and well-being of patients.

Meteorin is an endogenous protein which has previously been demonstrated to be a promote outgrowth of cultured neurons (WO 2005/095450). In addition, Meteorin has previously been shown to be effective in reversing neuropathic pain arising from peripheral nerve injury (WO 2012/041328).

### SUMMARY

The present invention provides means for improving quality of life for patients suffering from acute and chronic nociceptive pain. The inventors of the present disclosure have found that administration of Meteorin is an effective therapeutic strategy for management of nociceptive pain. Meteorin has been shown to possess both robust and prolonged analgesic actions, while being well tolerated. Meteorin fully reverses mechanical hyperalgesia in subjects suffering from inflammatory hyperalgesia, and analgesic tolerance does not occur with repeated administration.

In one aspect, the present invention relates to an isolated polypeptide for use in treatment and/or prevention of nociceptive pain in a subject, said polypeptide comprising an amino acid sequence selected from the group consisting of:
a. the amino acid sequence of SEQ ID NO: 3; and
b. a biologically active sequence variant of the amino acid sequence of SEQ ID NO: 3, wherein the variant has at least 70% sequence identity to SEQ ID NO: 3.

In a second aspect, the present invention relates to an isolated nucleic acid molecule for use in treatment and/or prevention of nociceptive pain in a subject, said nucleic acid molecule comprising a nucleic acid sequence coding for a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3.

In a further aspect, the present invention relates to a vector for use in treatment or prevention of nociceptive pain in a subject, said vector comprising a polynucleotide coding for a polypeptide for use in treatment and/or prevention of nociceptive pain in a subject

### DESCRIPTION OF DRAWINGS

FIG. 1: General study design using CFA inflammatory pain model. Mechanical thresholds were assessed using von Frey filaments (VF; solid arrows) at baseline (BL) and then from Days 3-5 after hindpaw injection of complete Freund's adjuvant (CFA; dashed circle). Once hyperalgesia was fully established, recombinant mouse rmMeteorin 1.8 mg/kg or Vehicle was administered s.c. to mice on Days 5, 7, 9, 11 and 13 in a first experiment (Example 1), and Days 3 (1 injection), Days 3 and 5 (2 injections), and Days 3, 5 and 7 (3 injections) in a second experiment (Example 2). Abbreviations; subcutaneous (s.c.).

FIG. 2. Repeated treatment with rmMeteorin completely reverses mechanical pain in mice with CFA inflammatory hyperalgesia. a) Hindpaw withdrawal thresholds (g) to von Frey stimulation were assessed in female C57BL/6JRj mice prior to hindpaw CFA (20 μl, s.c.) injection (dashed arrow) at baseline (BL) and then routinely afterwards until day 15 post-CFA injection. (b, c) Paw width (mm) and body weight (g) was measured prior to and routinely after CFA injection (dashed arrow) as surrogate markers of inflammatory load and general welfare respectively. Repeated systemic injection of rmMeteorin (1.8 mg/kg, s.c.) was performed on Days 5-13 (solid arrows) and produced a robust reversal of mechanical pain. In contrast, no effect on paw width or body weight was observed. Naïve mice were included for purposes of obtaining trunk blood samples for exposure analysis at study end. *P<0.05, **P<0.01, two-way RM ANOVA and Tukey's. Data are means±S.E.M.

FIG. 3. Acute treatment with rmMeteorin reverses mechanical pain similarly to repeated treatment in mice with CFA inflammatory hyperalgesia. a) Hindpaw withdrawal thresholds (g) to von Frey stimulation were assessed in female C57BL/6JRj mice prior to hindpaw CFA (20 μl, s.c.) injection (dashed arrow) at baseline (BL) and then routinely afterwards until day 14 post-CFA injection. Systemic injections of rmMeteorin (1.8 mg/kg, s.c.) were administered to 3 separate groups of mice on Days 3 (Group 1 (Meteorin1): 1 injection), Days 3 and 5 (Group 2 (Meteorin2): 2 injections), and Days 3, 5 and 7 (Group 3 (Meteorin3): 3 injections) as indicated by solid arrowheads. A single injection of rmMeteorin produced a similar magnitude and duration of reversal of mechanical hyperalgesia. b) At the end of the experiment on Days 14-15 the partial μ-opioid receptor agonist buprenorphine (0.1 mg/kg, s.c.) or Vehicle was administered to mice previously treated with repeated injections of Vehicle and effects on mechanical withdrawal thresholds assessed. The number of mice in each group is indicated in parentheses.

^{\hat{ }, #,} *P<0.05, ^{##,} **P<0.01, ^{\hat{ }\hat{ }\hat{ }}P<0.001, ^{\hat{ }\hat{ }\hat{ }\hat{ }}P<0.0001 vs. Corresponding Vehicle, two-way RM ANOVA and Tukey's. (b) ****P<0.0001 vs. Vehicle, Student's t test. Data are means±S.E.M.

FIG. 4: CLUSTAL W (1.82) multiple sequence alignment of Meteorin.

A) Alignment of Meteorin precursors from human (SEQ ID NO: 2), rat (SEQ ID NO: 9), and mouse (SEQ ID NO: 5). B) Alignment of mature Meteorin from human (SEQ ID NO: 3), rat (SEQ ID NO: 10), and mouse (SEQ ID NO: 6). C) Mature Meteorin, consensus sequence (SEQ ID NO: 11) generated from fully conserved residues in the human, mouse and rat sequences. X represents any of the 21 naturally occurring amino acids encoded by DNA.

### DETAILED DESCRIPTION

### Definitions

As used herein “a biocompatible capsule” means that the capsule, upon implantation in a host mammal, does not elicit a detrimental host response sufficient to result in the rejection of the capsule or to render it inoperable, for example through degradation.

As used herein, a “coding sequence” is a polynucleotide sequence which is transcribed and translated into a polypeptide.

As used herein, the term “expression vectors” refers to vectors that are capable of directing the expression of genes to which they are operably-linked. In general, expression vectors of utility using recombinant DNA techniques are often in the form of plasmids.

“Meteorin”, as used herein, refers to polypeptides having the amino acid sequences of substantially purified Meteorin obtained from any species, particularly mammalian, including chimpanzee, bovine, ovine, porcine, murine, equine, and preferably human, from any source whether natural, synthetic, semi-synthetic, or recombinant. The term also refers to biologically active fragments of Meteorin obtained from any of these species, as well as to biologically active sequence variants of these and to proteins subject to posttranslational modifications.

As used herein, the term “operably-linked” is intended to mean that the nucleotide sequence of interest is linked to the regulatory sequence(s) within a recombinant expression vector, in a manner that allows for expression of the nucleotide sequence (e.g., in an in vitro transcription/translation system or in a host cell when the vector is introduced into the host cell).

As used herein, the term “regulatory sequence” is intended to include promoters, enhancers and other expression control elements (e.g., polyadenylation signals).

“Sequence identity”: A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. The ClustalW software is available as a ClustalW WWW Service at the European Bioinformatics Institute from http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similarly be used to align nucleotide sequences. Sequence identities may be calculated in a similar way as indicated for amino acid sequences.

The term “subject” used herein is taken to mean any mammal to which Meteorin polypeptide or polynucleotide, therapeutic cells or biocompatible capsules may be administered. Subjects specifically intended for treatment with the method of the invention include humans, as well as nonhuman primates, sheep, horses, cattle, goats, pigs, dogs, cats, rabbits, guinea pigs, hamsters, gerbils, rats and mice.

“Treatment” can be performed in different ways, including curative and/or ameliorating. Curative treatment generally aims at curing a clinical condition, which is already present in the treated individual. Ameliorating treatment generally means treating in order to improve, in an individual, an existing clinical condition.

The term “prevention” as used herein refers to preventing a clinical condition or reducing the risk of contracting the condition or reducing the extent of the condition.

Prevention may also be referred to herein as prophylactic treatment or pre-emptive treatment.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Nociceptive Pain

Two main types of pain exist: nociceptive pain where the nerve system is intact and neuropathic pain, which arises due to injuries to the nerve system.

As used herein the term “neuropathic pain”, refers to pain caused by damage (lesion or disease) to the sensory nerves of the somatosensory nervous system (both the peripheral and the central nervous system), as also described by the International Association for the Study of Pain, IASP: https://www.iasp-pain.org/resources/terminology/#neuropathic-pain. Neuropathic pain is typically well localized, constant, and often with an aching or throbbing quality.

As used herein the term “nociceptive pain” refers to pain that arises from actual or threatened damage to non-neural tissue and is due to the activation of nociceptors, in accordance with the definition used by the IASP: https://www.iasp-pain.org/resources/terminology/#nociceptive-pain.

Nociceptive pain develops in response to a specific stimulus to the body and non-neural tissue of the body and informs the subject of impending tissue damage. Nociceptive pain includes tissue injury-induced pain and inflammatory pain.

IASP has recently revised their definition and terminology in relation to nociceptive pain and neuropathic pain to avoid any misinterpretation. IASP has added the following under the definition of nociceptive pain “This term is designed to contrast with neuropathic pain”. The revised definition emphasizes that the term nociceptive pain is designed to contrast with the term neuropathic pain.

As can be seen from the above, neuropathic pain and nociceptive pain are different phenomena with different underlying causes. Nociceptive pain describes pain occurring with a normally functioning somatosensory nervous system in contrast to the abnormal nerve function seen in neuropathic pain.

Common examples of nociceptive pain includes lower back pain, shoulder pain, musculoskeletal pain, arthritis pain, joint pain, post-operative pain, post-traumatic pain and cancer pain.

Nociceptive pain can be classified as being either visceral or somatic.

“Visceral pain” refers to pain which arises from the visceral organs, such as the gastrointestinal tract or pancreas.

“Somatic pain” refers to pain which arises from the musculoskeletal system, such as the skin, subcutaneous tissues, muscles, and joints.

Further nociceptive pain can both be acute and chronic.

“Acute pain” as used herein refers to sudden, severe pain from a specific cause (such as injury, infection, inflammation) that lasts a limited period of time.

“Chronic pain” as used herein refers to a persistent state of pain whereby the cause of the pain cannot be easily removed. Chronic pain can be constant or intermittent. Chronic pain may be defined as pain lasting more than a given time period, typically about three months. Chronic pain is often associated with long-term incurable or intractable medical conditions or diseases.

Common causes of chronic pain include, but are not limited to, arthritis, cancer, repetitive stress injuries, headaches, lower back pain, neck and shoulder pain, post-traumatic pain, postsurgical pain, moderate to severe osteoarthritis, and severe migraine.

As used herein, the term “inflammatory pain” refers to pain associated with inflammation, which is characterized by redness, swelling, warmth and pain. Inflammation is the nonspecific immune response of an organism to infection, irritation and/or injury and involves the release of pro-inflammatory molecules (e.g. peptides, cytokines, prostanoids, growth factors). These molecules sensitize the afferent terminals of peripheral sensory neurons which are involved in the transduction and transmission of stimuli such as touch, heat, cold and chemical information. Inflammatory pain often result in inflammatory hyperalgesia.

### Hyperalgesia

Hyperalgesia is an extreme response to a stimulus which is normally perceived as painful. The stimulus can be mechanical/tactile, thermal or chemical in origin. Hyperalgesia is often associated with nerve damage (neuropathic pain), however, hyperalgesia as used herein refers to an increased sensitivity caused by tissue injury or inflammation.

“Inflammatory hyperalgesia” as used herein refers to increased pain sensitivity that occurs directly in damaged tissues and pain sensitivity that occurs in surrounding undamaged tissues.

In one embodiment the present invention relates to the use of Meteorin in the treatment of hyperalgesia. In one embodiment of the present invention the hyperalgesia to be treated is inflammatory hyperalgesia.

In one embodiment of the present invention the hyperalgesia to be treated is mechanical hyperalgesia In another embodiment the hyperalgesia to be treated is thermal hyperalgesia.

In one embodiment of the present invention the thermal hyperalgesia is cold hyperalgesia. In another embodiment the thermal hyperalgesia is heat hyperalgesia.

Environmental irritants such as phthalates and heavy metals including lead, aluminum and mercury, can disrupt the immune function thereby triggering increased inflammation production, leading to chemical hyperalgesia.

In one embodiment of the present invention the hyperalgesia is chemical hyperalgesia. In one embodiment the chemical hyperalgesia is triggered by phthalates, lead, aluminium, mercury and/or other environmental irritants.

As stated above substantially full reversal of normal sensory function was achieved in animals receiving Meteorin. It is thus conceivable that Meteorin can mediate full reversal of hyperalgesia in at least a subset of the treated subjects.

Nociceptive pain may be induced by different stimuli, including injuries and inflammation. Meteorin can be used for prevention or treatment of nociceptive pain, such as acute nociceptive pain, chronic nociceptive pain, visceral nociceptive pain, somatic nociceptive pain.

In one embodiment the nociceptive pain is one or more of inflammatory pain, lower back pain, shoulder pain, musculoskeletal pain, arthritis pain, joint pain, post-operative pain, post-traumatic pain, cancer pain and other nociceptive pains.

In one embodiment the nociceptive pain is developed in response to a specific stimulus to the body. Specific stimuli include injuries to the body and tissue damage related to but not limited to inflammation, surgery, physical trauma, arthritis, cancer, repetitive stress injuries, headaches, moderate to severe osteoarthritis, and migraine.

In one embodiment the nociceptive pain is associated with inflammation, sensitizing the afferent terminals of peripheral sensory neurons involved in the transduction and transmission of stimuli such as touch, heat, cold and chemical information.

In one embodiment the nociceptive pain is associated with arthritis.

In one embodiment Meteorin is administered by intermittent administration.

In one embodiment Meteorin reverses inflammatory hyperalgesia within days of administration.

Treatment and/or Prevention of Nociceptive Pain

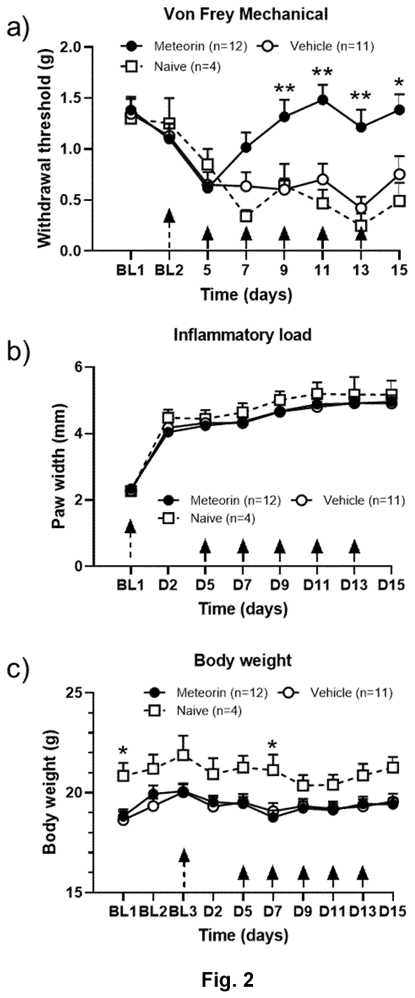

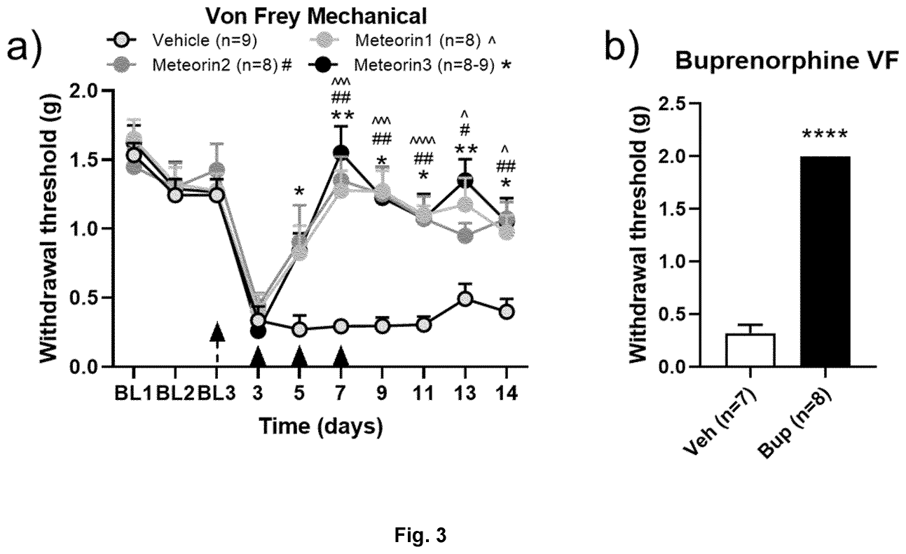

In example 1 it is demonstrated that continued intermittent administration of Meteorin completely reverses CFA-induced inflammatory hyperalgesia within days after initiation of dosing (FIG. 2). Further it is demonstrated, that the size of the inflammatory oedema is completely unaffected by rmMeteorin treatment. rmMeteorin had no effect on inflammatory load which indicates that it does not possess a direct anti-inflammatory mechanism per se.

In example 2 it is demonstrated that acute treatment (single injection) with rmMeteorin reverses mechanical pain similarly to repeated treatment (multiple injections) in mice with CFA inflammatory hyperalgesia. A single injection of rmMeteorin produced a similar magnitude and duration of reversal of hyperalgesia as that observed with three injections (FIG. 3).

Development of safe and effective therapies to prevent or treat nociceptive pain is desired, as drugs normally effective against chronic pain conditions, such as opioids, are associated with numerous side effects.

The present invention provides treatment, amelioration and/or prevention of nociceptive pain by administration of Meteorin to the subject in pain. Thus, in one embodiment the present invention relates to Meteorin for use in the treatment and/or prevention of nociceptive pain. In one embodiment the present invention relates to Meteorin for use in the treatment of nociceptive pain.

Meteorin can be used for prevention and treatment of acute nociceptive pain. In one embodiment Meteorin is used for treatment of acute nociceptive pain. Further Meteorin can be used for treatment and prevention of chronic nociceptive pain. In one embodiment Meteorin is used for treatment of chronic nociceptive pain

In one embodiment, the present disclosure provides an isolated polypeptide for use in treatment and/or prevention of nociceptive pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one embodiment, the present invention relates to a method for treatment and/or prevention of nociceptive pain, the method comprising administering a therapeutically effective amount of an isolated polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3,
   to a subject in need thereof.

In one embodiment, the present disclosure provides use of an isolated polypeptide for the manufacture of a medicament for the treatment and/or prevention of nociceptive pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one embodiment the present invention relates to the use of Meteorin in a method of treatment of nociceptive hyperalgesia. In another embodiment the present invention relates to the use of Meteorin for the treatment of mechanical nociceptive hyperalgesia. In one embodiment the present invention relates to the use of Meteorin for the treatment of thermal nociceptive hyperalgesia. In another embodiment the present invention relates to the use of Meteorin for the treatment of cold nociceptive hyperalgesia. In another embodiment the present invention relates to the use of Meteorin for treatment of heat nociceptive hyperalgesia. In yet another embodiment the present invention relates to the use of Meteorin for the treatment of chemical hyperalgesia.

In one embodiment the present invention relates to the use of Meteorin for the treatment of nociceptive pain. In a more preferred embodiment the present invention relates to the use of Meteorin for the treatment of pain associated with inflammation or injuries to the body. In one embodiment Meteorin is used in a method reversing mechanical pain in subjects suffering from inflammatory hyperalgesia.

As demonstrated in examples 1 and 2 of the present disclosure, administration of Meteorin fully diminishes gain of sensory function in subjects suffering from inflammatory hyperalgesia. The reversal of inflammatory hyperalgesia can be maintained as analgesic tolerance does not occur with Meteorin.

Thus, in a preferred embodiment the present invention relates to Meteorin for use in prevention and/or treatment of nociceptive pain.

In one embodiment, the present disclosure provides an isolated polypeptide for use in prevention and/or treatment of nociceptive pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one aspect the present disclosure relates to an isolated polypeptide is for use in prevention and/or treatment of nociceptive pain in a subject.

In one embodiment the present disclosure relates to an isolated polypeptide is for use in prevention and/or treatment of nociceptive pain in a subject. In one embodiment nociceptive pain is somatic pain or visceral pain. In another embodiment nociceptive pain is inflammatory pain, lower back pain, shoulder pain, musculoskeletal pain, arthritis pain, joint pain, post-operative pain, post-traumatic pain or cancer pain.

In another embodiment wherein the nociceptive pain is selected from the group consisting of inflammatory pain and post-operative pain.

In one embodiment of the present disclosure the isolated polypeptide is for use in prevention and/or treatment of nociceptive pain, wherein the nociceptive pain is nociceptive hyperalgesia or inflammatory pain, such as inflammatory hyperalgesia. In another embodiment of the present disclosure the isolated polypeptide is for use in prevention and/or treatment of chemical hyperalgesia.

In one embodiment of the present disclosure the isolated polypeptide is for use in prevention and/or treatment of nociceptive pain, wherein the nociceptive pain is post-operative pain.

In one embodiment of the present disclosure the isolated polypeptide is for use in prevention and/or treatment of nociceptive pain, wherein the subject suffers from a disease or disorder selected from the group consisting of arthritis, inflammatory pain, and post-operative pain.

In one embodiment arthritis is selected from the group consisting of Osteoarthritis, Rheumatoid arthritis, or Lupus.

In another embodiment inflammatory pain is selected from the group consisting of inflammatory hyperalgesia, post-operative pain and arthritis.

In one embodiment, the present invention relates to a method of treatment of nociceptive pain, the method comprising administering a therapeutically effective amount of an isolated polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3,
   to a subject in need thereof.

In one embodiment, the present disclosure provides use of an isolated polypeptide for the manufacture of a medicament for use in prevention and/or treatment of nociceptive pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

### Administration and Formulation

Meteorin polypeptides may be administered in any manner, which is medically acceptable. This may include injections, by parenteral routes such as intravenous, intravascular, intraarterial, subcutaneous, intramuscular, intratumor, intraperitoneal, intraventricular, intraepidural, intrathecal, intracerebroventricular, intercerebral, or others as well as nasal, or topical. Slow-release administration is also specifically included in the invention, by such means as depot injections or erodible implants.

Administration of Meteorin according to this invention may be achieved using any suitable delivery means, including: injection, either subcutaneously, intravenously, intra-arterially, intramuscularly, intrathecally or to other suitable site; pump (see, e.g., Annals of Pharmacotherapy, 27:912 (1993); Cancer, 41:1270 (1993); Cancer Research, 44:1698 (1984), incorporated herein by reference); microencapsulation (see, e.g., U.S. Pat. Nos. 4,352,883; 4,353,888; and 5,084,350, herein incorporated by reference), slow release polymer implants (see, e.g., Sabel, U.S. Pat. No. 4,883,666, incorporated herein by reference); encapsulated cells (see, “Biocompatible capsules”); unencapsulated cell grafts (see, e.g., U.S. Pat. Nos. 5,082,670 and 5,618,531, each incorporated herein by reference); and inhalation.

Administration may be by periodic injections of a bolus of the preparation or may be made more continuous by intravenous or intraperitoneal administration from a reservoir which is external (e.g., an IV bag) or internal (e.g., a bioerodable implant, a bioartificial organ, a biocompatible capsule of Meteorin production cells, or a colony of implanted Meteorin production cells). See, e.g., U.S. Pat. Nos. 4,407,957, 5,798,113, and 5,800,828, each incorporated herein by reference.

Localised delivery may be by such means as delivery via a catheter to one or more arteries. In one embodiment of the present invention localised delivery comprises delivery using encapsulated cells (as described in the section “biocompatible capsule”).

A further type of localised delivery comprises local delivery of gene therapy vectors, which are normally injected.

In a preferred embodiment of the present invention the administration is parenteral injection, preferably subcutaneous injection, or intrathecal injection.

Whilst it is possible for the compounds of the present invention to be administered as the raw chemical, it is preferred to present them in the form of a pharmaceutical formulation. The pharmaceutical formulations may be prepared by conventional techniques, e.g. as described in Remington: The Science and Practice of Pharmacy 2005, Lippincott, Williams & Wilkins.

The term “pharmaceutically acceptable carrier” means one or more organic or inorganic ingredients, natural or synthetic, with which Meteorin polypeptide is combined to facilitate its application. A suitable carrier includes sterile saline although other aqueous and non-aqueous isotonic sterile solutions and sterile suspensions known to be pharmaceutically acceptable are known to those of ordinary skill in the art.

The compounds of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. A recombinant Meteorin protein is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Where slow-release administration of a Meteorin polypeptide is desired in a formulation with release characteristics suitable for the treatment of any disease or disorder requiring administration of a Meteorin polypeptide, microencapsulation of a Meteorin polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The slow-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

In one embodiment of the present invention a composition comprising Meteorin is contemplated. The composition may comprise an isolated polypeptide as described herein, an isolated nucleic acid as described herein, a Meteorin encoding expression vector as described herein, a cell line expressing Meteorin as described herein or a biocompatible capsule secreting Meteorin as described herein.

### Dosages

Various dosing regimens for systemic administration are contemplated. In one embodiment, methods of administering to a subject a formulation comprising a Meteorin polypeptide include administering Meteorin at a dosage of between 1 μg/kg and 10,000 μg/kg body weight of the subject, per dose. In another embodiment, the dosage is between 1 μg/kg and 7,500 μg/kg body weight of the subject, per dose. In a further embodiment, the dosage is between 1 μg/kg and 5,000 μg/kg body weight of the subject, per dose. In a different embodiment, the dosage is between 1 μg/kg and 2,000 μg/kg body weight of the subject, per dose. In yet another embodiment, the dosage is between 1 μg/kg and 1,000 μg/kg body weight of the subject, per dose. In yet another embodiment, the dosage is between 1 μg/kg and 700 μg/kg body weight of the subject, per dose. In a more preferable embodiment, the dosage is between 5 μg/kg and 500 μg/kg body weight of the subject, per dose. In a most preferable embodiment, the dosage is between 10 μg/kg and 100 μg/kg body weight of the subject, per dose. In a preferred embodiment the subject to be treated is human.

Guidance as to particular dosages and methods of delivery is provided in the literature, see, for example, WO 02/78730 and WO 07/100898. Guidance to the calculation of the human equivalent dosages based on dosages used in animal experiments is provided in Reagan-Shaw et al., FASEB J, 22, 659-661 (2007).

The dose administered must be carefully adjusted to the age, weight and condition of the individual being treated, as well as the route of administration, dosage form and regimen, and the result desired, and the exact dosage should be determined by the practitioner.

In one embodiment of the present invention Meteorin is administrated by systemic administration.

In one embodiment Meteorin is administered by parenteral injection, preferably subcutaneous injection or intrathecal injection.

In one embodiment Meteorin polypeptide is administered in dosages of 1 μg/kg-10,000 μg/kg, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body.

In one embodiment of the present invention the administration is repeated daily. In another embodiment the administration is repeated at least 1-3 times weekly, such as 2-5 times weekly, such as 3-6 times weekly.

In one embodiment, the administration is repeated once a day, once every two days, once every three days, once every four days, once every five days, once every six days, or once every 7 days. In a preferred embodiment, the administration is repeated once every two days.

In one embodiment, the present invention provides treatment of nociceptive pain. Thus, in one embodiment, the administration is initiated after onset of symptoms of nociceptive pain.

In one embodiment, the present invention provides prevention of nociceptive pain. Thus, in one embodiment, the Meteorin polypeptide is administered prior to onset of symptoms of nociceptive pain.

In other embodiments, Meteorin is administered at relatively long dosage interval. A relatively long dosage interval is intended to include at least 2 days between dosages, such as at least 3 days between dosages, for example 2 dosages per week. More preferably the long dosages intervals are at least one week, such as at least 2 weeks, more preferably at least 3 weeks, such as at least 4 weeks, or at least one month.

By a relatively long dosage interval is intended at least 2 days between dosages, such as at least 3 days between dosages, for example 2 dosages per week. More preferably the long dosages interval is at least one week, such as at least 2 weeks, more preferably at least 3 weeks, such as at least 4 weeks, or at least one month.

Expressed in a different way the dosage intervals are so long that following one dosage of Meteorin polypeptide, the polypeptide is no longer detectable in the serum of the subject to be treated when the next dosage is administered. In another embodiment the blood serum level is below 10 ng/mL, such as below 5 ng/mL, more preferably below 1 ng/mL, such as below 0.5 ng/mL, for example below 0.1 ng/mL.

In some embodiments, the long dosage range is preceded by more frequent initial administration of Meteorin, e.g., twice daily, daily, once every two days, once every three days, or once every four days. This initial dosing schedule may be maintained e.g., for 2, 3, 4, 5, 6, 7, 9, 11, 14, 21 days, or more. After completion of this dosing schedule, Meteorin can be administered less frequently, e.g., as described above.

Thus in one aspect, the invention relates to a method of treating neuropathic pain in a human subject in need thereof comprising administering to the subject a therapeutically effective amount of a neurotrophic polypeptide comprising an amino acid sequence having at least 70% identity to the amino acid sequence of SEQ ID NO: 3, wherein said administration is three times per week or more infrequently.

Preferably, the administration is weekly or more infrequent administration. Even more preferably the administration is bi-weekly or more infrequent administration.

Expressed in a different way the dosage intervals are so long that following one dosage of Meteorin polypeptide, the polypeptide is no longer detectable in the serum of the subject to be treated when the next dosage is administered. In another embodiment the blood serum level is below 10 ng/mL, such as below 5 ng/mL, more preferably below 1 ng/mL, such as below 0.5 ng/mL, for example below 0.1 ng/mL.

In some embodiments, the initial administration of Meteorin is, e.g., twice daily, daily, once every two days, once every three days, or once every four days. This dosing schedule may be maintained e.g., for 2, 3, 4, 5, 6, 7, 9, 11, 14, 21 days, or more. After completion of this dosing schedule, Meteorin can be administered less frequently, e.g., as described above.

### Meteorin

The present invention relates to the use of polypeptides being identified as Meteorin protein and polynucleotides encoding said protein, in the treatment of nociceptive pain. The delivery is in one embodiment contemplated to be by use of a capsule for delivery of a secreted biologically active Meteorin and/or a homologue thereof to a subject. The Meteorin protein has been identified in human beings (SEQ ID NO: 2), mouse (SEQ ID NO: 5), and rat (SEQ ID NO: 8) and a variety of other species.

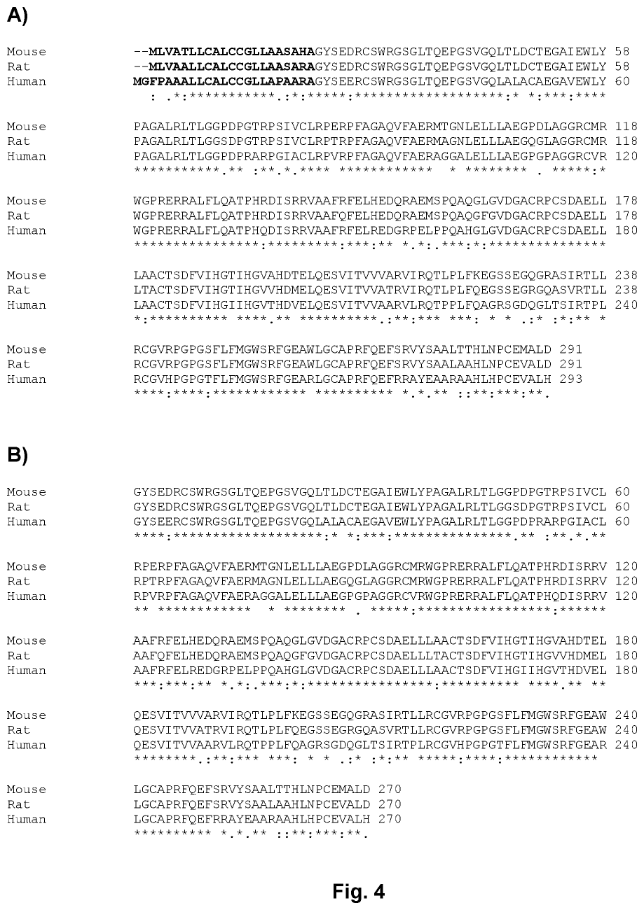

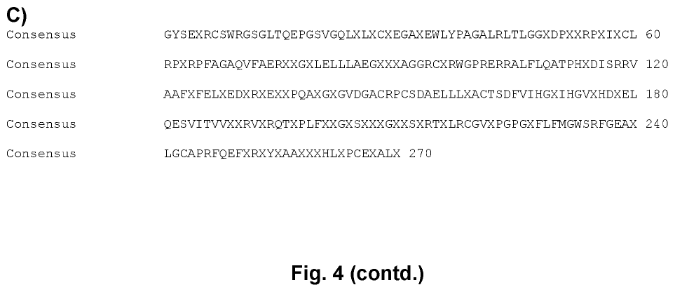

Human Meteorin exists as a 293 amino acid precursor, which can be processed to give rise to at least one biologically active peptide. Meteorin is expressed at high levels in the nervous system and the eye, and in particular subregions of the brain. The mouse (SEQ ID NO: 5) and rat (SEQ ID NO: 8) Meteorin precursors consist of 291 amino acids, and the % sequence identities with the human Meteorin protein (SEQ ID NO: 2) are 80.3 and 80.2, respectively (See FIG. 4).

Human Meteorin contains an N-terminal signal peptide sequence of 23 amino acids, which is cleaved at the sequence motif ARA-GY. This signal peptide cleavage site is predicted by the SignalP method. The N-terminal of mouse Meteorin has been verified by N-terminal sequencing (Jørgensen et al., Characterization of Meteorin—An evolutionary conserved neurotrophic factor, J mol Neurosci 2009 September; 39 (1-2): 104-116).

Table 1 shows the % sequence identity between full length human Meteorin versus mouse and rat sequences. See alignment in FIG. 4a.

| Sequence | % sequence identity |
|---|---|
| human | — |
| mouse | 80.3 |
| rat | 80.2 |

Table 2 shows the % sequence identity between human Meteorin versus mouse and rat sequences after removal of N-terminal signal peptide. See alignment in FIG. 4b.

| Sequence | % sequence identity |
|---|---|
| human | — |
| mouse | 81.9 |
| rat | 79.6 |

Based on the fully conserved residues, a consensus sequence for mature Meteorin can be derived (SEQ ID NO: 11, FIG. 4c), wherein X is independently selected from any of the 21 naturally occurring amino acid encoded by DNA. In a preferred embodiment a variant Meteorin comprises the consensus sequence.

One biological function of Meteorin is the ability to induce neurite outgrowth in dissociated dorsal root ganglia (DRG) cultures as described in Jørgensen et al., Characterization of Meteorin—An evolutionary conserved neurotrophic factor, J mol Neurosci 2009 September; 39 (1-2): 104-116 and Nishino et al., “Meteorin: a secreted protein that regulates glial cell differentiation and promotes axonal extension”, EMBO J., 23(9):1998-2008 (2004).

Due to the high conservation of the cysteines, it is expected that these residues play an important role in the secondary and tertiary structure of the bioactive protein. One or more of the cysteines may participate in the formation of intra- and/or intermolecular disulfide bridges.

### Meteorin Polypeptides

In addition to full-length Meteorin, substantially full-length Meteorin, and to pro-Meteorin, the present invention provides for biologically active variants of the polypeptides. A Meteorin polypeptide or fragment is biologically active if it exhibits a biological activity of naturally occurring Meteorin as described herein, such as being neurotrophic. It is to be understood that the invention relates to Meteorin as herein defined.

The invention relates to an isolated polypeptide molecule for use in a method of treatment of nociceptive pain, said polypeptide comprising an amino acid sequence selected from the group consisting of:
a) the amino acid sequence selected from the group consisting of SEQ ID NO: 3, 6 and 9;
b) a biologically active sequence variant of the amino acid sequence selected from the group consisting of SEQ ID NO: 3, 6 and 9, wherein the variant has at least 70% sequence identity to said SEQ ID NO; and
c) a biologically active fragment of at least 50 contiguous amino acids of any of a) or b) wherein the fragment is at least 70% identical to said SEQ ID NO.

In one embodiment the invention relates to an isolated polypeptide selected from the group consisting of:
i) AA₃₀-AA₂₈₈ of SEQ ID NO: 2, and polypeptides having from one to five extra amino acids from the native sequence in one or both ends, up to AA₂₅-AA₂₉₃ of SEQ ID NO: 2;
ii) AA₂₈-AA₂₈₆ of SEQ ID NO: 8 and polypeptides having from one to five extra amino acids from the native sequence in one or both ends, up to AA₂₃-AA₂₉₁ of SEQ ID NO: 8;
iii) AA₃₁-AA₂₈₉ of SEQ ID NO: 5 and polypeptides having from one to five extra amino acids from the native sequence in one or both ends, up to AA₂₆-AA₂₉₄ of SEQ ID NO: 5; and
iv) variants of said polypeptides, wherein any amino acid specified in the chosen sequence is changed to a different amino acid, provided that no more than 20 of the amino acid residues in the sequence are so changed.

A preferred biological activity is the ability to elicit substantially the same response as in the DRG assay as obtained for mouse Meteorin described in Jørgensen et al., Characterization of Meteorin—An evolutionary conserved neurotrophic factor, J mol Neurosci 2009 September; 39 (1-2): 104-116. In this assay DRG cells are grown in the presence of full length human Meteorin coding sequence (SEQ ID NO: 3). By substantially the same response in the DRG assay is intended that the neurite outgrowth from DRG cells is at least 20% of the number obtained in the DRG assay described in Jørgensen et al., Characterization of Meteorin—An evolutionary conserved neurotrophic factor, J mol Neurosci 2009 September; 39 (1-2): 104-116, more preferably at least 30%, more preferably at least 40%, more preferably at least 50%, more preferably at least 60%, more preferably at least 70%, more preferably at least 75%, more preferably at least 80%, more preferably at least 85%, more preferably at least 90%. The biological activity of a fragment or variant of Meteorin may also be higher than that of the naturally occurring Meteorin (SEQ ID NO: 3).

Variants can differ from naturally occurring Meteorin in amino acid sequence or in ways that do not involve sequence, or in both ways. Variants in amino acid sequence (“sequence variants”) are produced when one or more amino acids in naturally occurring Meteorin is substituted with a different natural amino acid, an amino acid derivative or non-native amino acid. Particularly preferred variants include naturally occurring Meteorin, or biologically active fragments of naturally occurring Meteorin, whose sequences differ from the wild type sequence by one or more conservative and/or semi-conservative amino acid substitutions, which typically have minimal influence on the secondary and tertiary structure and hydrophobic nature of the protein or peptide. Variants may also have sequences, which differ by one or more non-conservative amino acid substitutions, deletions or insertions, which do not abolish the Meteorin biological activity. The Clustal W alignment in FIG. 4 can be used to predict which amino acid residues can be substituted without substantially affecting the biological acitivity of the protein. In a preferred embodiment a variant Meteorin sequence comprises the consensus sequence having SEQ ID NO: 11.

Substitutions within the following group (Clustal W, ‘strong’ conservation group) are to be regarded as conservative substitutions within the meaning of the present invention -S,T,A; N,E,Q,K; N,H,Q,K; N,D,E,Q; Q,H,R,K; M,I,L,V; M,I,L,F; H,Y; F,Y,W.

Substitutions within the following group (Clustal W, ‘weak’ conservation group) are to be regarded as semi-conservative substitutions within the meaning of the present invention -C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

Other variants within the invention are those with modifications which increase peptide stability. Such variants may contain, for example, one or more nonpeptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D-amino acids instead of L-amino acids into the polypeptide may increase its resistance to proteases. See, e. g., U.S. Pat. No. 5,219,990. Splice variants are specifically included in the invention.

When the result of a given substitution cannot be predicted with certainty, the derivatives may be readily assayed according to the methods disclosed herein to determine the presence or absence of neurotrophic activity, preferably using the DRG assay described in Jørgensen et al., Characterization of meteorin—An evolutionary conserved neurotrophic factor, J mol Neurosci 2009 September; 39 (1-2): 104-116.

In one embodiment, the polypeptide is a naturally occurring allelic variant of the sequence selected from the group consisting of SEQ ID NO: 3, 6 and 9. This polypeptide may comprise an amino acid sequence that is the translation of a nucleic acid sequence differing by a single nucleotide from a nucleic acid sequence selected from the group consisting of SEQ ID NO: 1, 4 and 7.

A variant polypeptide as described herein, in one embodiment comprises a polypeptide wherein any amino acid specified in the chosen sequence is changed to provide a conservative substitution.

Variants within the scope of the invention in one embodiment include proteins and peptides with amino acid sequences having at least 70 percent identity with human, murine or rat Meteorin (SEQ ID NO: 3, 6, and 9). More preferably the sequence identity is at least 75%, more preferably at least 80%, more preferably at least 85%, more preferably at least 90%, more preferably at least 95%, more preferably at least 98%.

In a preferred embodiment the sequence identity of the variant Meteorin is determined with reference to a human Meteorin polypeptide (SEQ ID NO: 3).

In one embodiment, the variants include proteins comprising an amino acid sequence having at least 70% sequence identity to SEQ ID NO: 3, more preferably at least 75%, more preferably at least 80%, more preferably at least 85%, more preferably at least 90%, more preferably at least 95%, more preferably at least 98%.

In one embodiment, preferred variants include proteins comprising an amino acid sequence having at least 70% sequence identity to SEQ ID NO: 6, more preferably at least 75%, more preferably at least 80%, more preferably at least 85%, more preferably at least 90%, more preferably at least 95%, more preferably at least 98%.

In one embodiment, preferred variants include proteins comprising an amino acid sequence having at least 70% sequence identity to SEQ ID NO: 9, more preferably at least 75%, more preferably at least 80%, more preferably at least 85%, more preferably at least 90%, more preferably at least 95%, more preferably at least 98%.

The neurotrophic polypeptide preferably has at least 85% sequence identity to the amino acid sequence of SEQ ID NO: 3, more preferably at least 90%, more preferably at least 95%, more preferably at least 98%.

In one embodiment the neurotrophic polypeptide comprises the consensus sequence of SEQ ID NO: 11.

Preferably the neurotrophic polypeptide has cysteine residues at positions 7, 28, 59, 95, 148, 151, 161, 219, 243, and 265 relative to the amino acid sequence of SEQ ID NO: 3. In one embodiment, preferred variants of Meteorin include proteins comprising 50-270 amino acids, more preferably 75-270 amino acids, more preferably 90-270 amino acids, more preferably 100-270 amino acids, more preferably 125-270 amino acids, more preferably 150-270 amino acids, more preferably 175-270 amino acids, more preferably 200-270 amino acids, more preferably 225-270 amino acids, more preferably 250-270 amino acids.

In one embodiment, a variant Meteorin at corresponding positions comprises the residues marked in FIG. 4 as fully conserved (*), more preferably a variant Meteorin also comprises at corresponding positions the residues marked in FIG. 4 as strongly conserved (: strongly conserved groups include: S,T,A; N,E,Q,K; N,H,Q,K; N,D,E,Q; Q,H,R,K; M,I,L,V; M,I,L,F; H,Y; F,Y,W), more preferably a variant Meteorin also comprises at corresponding positions the residues marked in FIG. 4 as less conserved (less conserved groups include: C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y). In particular, it is contemplated that the conserved cysteines must be located at corresponding positions in a variant Meteorin. Thus in one embodiment, a variant Meteorin sequence has cysteine residues at positions 7, 28, 59, 95, 148, 151, 161, 219, 243, and 265 relative to the amino acid sequence of SEQ ID NO: 3.

In one embodiment, the polypeptide for use in the treatment and/or prevention of nociceptive pain comprises the consensus sequence of SEQ ID NO:11.

In one embodiment the polypeptide for use in the treatment and/or prevention of nociceptive pain has cysteine residues at positions 7, 28, 59, 95, 148, 151, 161, 219, 243, and 265 relative to the amino acid sequence of SEQ ID NO:3.

In one embodiment the polypeptide for use in the treatment and/or prevention of nociceptive pain is a variant polypeptide, wherein any amino acid substitutions are conservative substitutions.

In one embodiment the polypeptide for use in the treatment and/or prevention of nociceptive pain is capable of forming at least one intramolecular disulfide bridge.

In one embodiment the polypeptide for use in the treatment and/or prevention of nociceptive pain in subjects such as mammalian, preferably primate, more preferably human.

In one embodiment the polypeptide for use in the treatment and/or prevention of nociceptive pain is administered by systemic administration, such as by parenteral injection, preferably subcutaneous injection or intrathecal injection.

In one embodiment the polypeptide for use in the treatment and/or prevention of nociceptive pain is administered in dosages of 1 μg/kg-10,000 μg/kg, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body.

In one embodiment the polypeptide for use in the treatment and/or prevention of nociceptive pain is administered at least 1-3 times weekly, such as 2-5 times weekly, such as 3-6 times weekly. In another embodiment the polypeptide is administrated every day. In another embodiment the polypeptide is administered daily.

In one embodiment the polypeptide for use in the treatment and/or prevention of nociceptive pain, the administration of said polypeptide is initiated after onset of symptoms of nociceptive pain.

In one embodiment the encoded polypeptide comprises the consensus sequence of SEQ ID NO:11. The consensus sequence comprises the amino acid residues conserved in human, mouse and rat Meteorin as shown in FIG. 4c Preferably the neurotrophic polypeptide has cysteine residues at positions 7, 28, 59, 95, 148, 151, 161, 219, 243, and 265 relative to the amino acid sequence of SEQ ID NO: 3.

Non-sequence modifications may include, for example, in vivo or in vitro chemical derivatisation of portions of naturally occurring Meteorin, as well as acetylation, methylation, phosphorylation, carboxylation, PEG-ylation, or glycosylation. Just as it is possible to replace substituents of the protein, it is also possible to substitute functional groups, which are bound to the protein with groups characterized by similar features. Such modifications do not alter primary sequence. These will initially be conservative, i.e., the replacement group will have approximately the same size, shape, hydrophobicity and charge as the original group.

Many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention are, to name an illustrative few, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance, I. E. Creighton, Proteins-Structure and Molecular Properties, 2nd Ed., W. H. Freeman and Company, New York, 1993. Many detailed reviews are available on this subject, such as, for example, those provided by Wold, F., in Posttranslational Covalent Modification of Proteins, B. C. Johnson, Ed., Academic Press, New York, pp 1-12, 1983; Seifter et al., Meth. Enzymol. 182: 626-646, 1990 and Rattan et al., Protein Synthesis: Posttranslational Modifications and Aging, Ann. N.Y. Acad. Sci. 663: 48-62, 1992.

In addition, the protein may comprise a protein tag to allow subsequent purification and optionally removal of the tag using an endopeptidase. The tag may also comprise a protease cleavage site to facilitate subsequent removal of the tag. Non-limiting examples of affinity tags include a polyhis tag, a GST tag, a HA tag, a Flag tag, a C-myc tag, a HSV tag, a V5 tag, a maltose binding protein tag, a cellulose binding domain tag. Preferably for production and purification, the tag is a polyhistag. Preferably, the tag is in the C-terminal portion of the protein.

The native signal sequence of Meteorin may also be replaced in order to increase secretion of the protein in recombinant production in other mammalian cell types.

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well.

The modifications that occur in a polypeptide often will be a function of how it is made. For polypeptides made by expressing a cloned gene in a host, for instance, the nature and extent of the modifications in large part will be determined by the host cell's posttranslational modification capacity and the modification signals present in the polypeptide amino acid sequence. For instance, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation, inter alia. Similar considerations apply to other modifications.

It will be appreciated that the same type of modification may be present to the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

### Meteorin Nucleotide Sequences

The invention provides medical use of genomic DNA and cDNA coding for Meteorin, including for example the human cDNA nucleotide sequence (SEQ ID NO: 1 and 10), the mouse cDNA sequences (SEQ ID NO: 4) and rat cDNA sequences (SEQ ID NO: 7).

Variants of these sequences are also included within the scope of the present invention.

The invention relates to an isolated nucleic acid molecule for use in a method of treatment and/or prevention of nociceptive pain, said nucleic acid molecule comprising a nucleic acid sequence encoding a polypeptide, said polypeptide comprising an amino acid sequence selected from the group consisting of:
i. The amino acid sequence of SEQ ID NO: 3;
ii. A biologically active sequence variant of the amino acid sequence of SEQ ID NO: 3, wherein the variant has at least 70% sequence identity to SEQ ID NO: 3; and
iii. A biologically active fragment of at least 50 contiguous amino acids of i) or ii) wherein the fragment is at least 70% identical to SEQ ID NO: 3.

In one aspect, the invention relates to an isolated nucleic acid molecule for use in treatment and/or prevention of nociceptive pain in a subject

In one embodiment the isolated nucleic acid molecule comprises a nucleic acid sequence coding for a polypeptide comprising an amino acid sequence selected from the group consisting of:
a. the amino acid sequence of SEQ ID NO: 3;
b. a biologically active sequence variant of the amino acid sequence of SEQ ID NO:3, wherein the variant has at least 70% sequence identity to SEQ ID NO:3.

In one embodiment the invention relates to an isolated nucleic acid molecule for use in a method of treatment and/or prevention of nociceptive pain encoding a polypeptide, said polypeptide comprising an amino acid sequence selected from the group consisting of:
i) AA₃₀-AA₂₈₈ of SEQ ID NO: 2, and polypeptides having from one to five extra amino acids from the native sequence in one or both ends, up to AA₂₅-AA₂₉₃ of SEQ ID NO: 2;
ii) AA₂₈-AA₂₈₆ of SEQ ID NO: 8 and polypeptides having from one to five extra amino acids from the native sequence in one or both ends, up to AA₂₃-AA₂₉₁ of SEQ ID NO: 8;
iii) AA₃₁-AA₂₈₉ of SEQ ID NO: 5 and polypeptides having from one to five extra amino acids from the native sequence in one or both ends, up to AA₂₆-AA₂₉₄ of SEQ ID NO: 5; and
iv) variants of said polypeptides, wherein any amino acid specified in the chosen sequence is changed to a different amino acid, provided that no more than 20 of the amino acid residues in the sequence are so changed.

The nucleic acid molecule may comprise the nucleotide sequence of a naturally occurring allelic nucleic acid variant.

The nucleic acid molecule of the invention may encode a variant polypeptide, wherein the variant polypeptide has the polypeptide sequence of a naturally occurring polypeptide variant.

In one embodiment the nucleic acid molecule differs by a single nucleotide from a nucleic acid sequence selected from the group consisting of SEQ ID NO: 1, 4, 7 and 10. Preferably the encoded polypeptide has at least 60% sequence identity to a sequence selected from the group consisting of SEQ ID NO: 3 preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at leat 98% sequence identiy, more preferably wherein the polypeptide has a sequence selected from the group consisting of said SEQ ID NOs. Said sequences constitute human Meteorin.

In a preferred embodiment, the encoded polypeptide comprises the consensus sequence having SEQ ID NO: 11.

In a preferred embodiment the encoded polypeptide has at least 70% sequence identity to SEQ ID NO: 3, more preferably at least 75%, more preferably at least 80%, more preferably at least 95%, more preferably at least 98%, more preferably wherein said polypeptide has the sequence of SEQ ID NO: 3.

In one aspect the nucleic acid molecule comprises a nucleotide sequence selected from the group consisting of
a) the nucleotide sequence selected from the group consisting of SEQ ID NO: 1, 4, 7 and 10;
b) a nucleotide sequence having at least 70% sequence identity to a nucleotide sequence selected from the group consisting of SEQ ID NO: 1, 4, 7 and 10; and
c) a nucleic acid sequence of at least 150 contiguous nucleotides of a sequence selected from the group consisting of SEQ ID NO: 1, 4, 7 and 10;

In one embodiment, the isolated polynucleotide of the invention has at least 60, more preferably at least 65%, more preferably at least 70%, more preferably at least 75%, more preferably at least 80%, preferably at least 85%, more preferred at least 90%, more preferred at least 95%, more preferred at least 98% sequence identity to the polynucleotide sequence presented as SEQ ID NO: 1.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence presented as SEQ ID NO: 10.

In one embodiment, preferred isolated polynucleotide variants of the invention comprises 150-900 nucleic acids, more preferably 175-900 nucleic acids, more preferably 200-900 nucleic acids, more preferably 225-900 nucleic acids, more preferably 250-900 nucleic acids, more preferably 300-900 nucleic acids, more preferably 350-900 nucleic acids, more preferably 400-900 nucleic acids, more preferably 450-900 nucleic acids, more preferably 500-900 nucleic acids, more preferably 550-900 nucleic acids, more preferably 600-900 nucleic acids, more preferably 650-900 nucleic acids, more preferably 700-900 nucleic acids, more preferably 750-900 nucleic acids, more preferably 800-900 nucleic acids, more preferably 850-900 nucleic acids.

A preferred group of isolated polynucleotides include SEQ ID NO: 1 and 10, which are human Meteorin cDNA sequences. Generally the cDNA sequence is much shorter than the genomic sequences are more easily inserted into an appropriate expression vector and transduced/fected into a production cell or a human cell in vivo or ex vivo.

In addition, the nucleotide sequences of the invention include sequences, which are derivatives of these sequences. The invention also includes vectors, liposomes and other carrier vehicles, which encompass one of these sequences or a derivative of one of these sequences. The invention also includes proteins transcribed and translated from Meteorin cDNA, preferably human Meteorin cDNA, including but not limited to human Meteorin and derivatives and variants.

Codon optimised nucleic acid molecules for enhanced expression in selected host cells, including but not limited to E. coli, yeast species, Chinese Hamster, Baby Hamster, insect, fungus, and human are also contemplated.

Variant nucleic acids can be made by state of the art mutagenesis methods. Methods for shuffling coding sequences from human with those of mouse, rat or chimpanzee are also contemplated.

Variant nucleic acids made by exchanging amino acids present in human Meteorin with the amino acid present in mouse or rat Meteorin at the corresponding position, should this amino acid be different from the one present in human Meteorin.

### Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV. A further group of suitable retroviruses includes the group consisting of HIV, SIV, FIV, EAIV, CIV. Another group of preferred virus vectors includes the group consisting of alphavirus, adenovirus, adeno associated virus, baculovirus, HSV, coronavirus, Bovine papilloma virus, Mo-MLV, preferably adeno associated virus.

Preferred viruses for treatment of disorders of the nervous system are lentiviruses and adeno-associated viruses. Both types of viruses can integrate into the genome without cell divisions, and both types have been tested in pre-clinical animal studies for indications of the nervous system, in particular the central nervous system.

Methods for preparation of AAV are described in the art, e.g. U.S. Pat. No. 5,677,158. U.S. Pat. Nos. 6,309,634 and 6,683,058 describe examples of delivery of AAV to the central nervous system.

Preferably, a lentivirus vector is a replication-defective lentivirus particle. Such a lentivirus particle can be produced from a lentiviral vector comprising a 5′ lentiviral LTR, a tRNA binding site, a packaging signal, a promoter operably-linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry 7-8 kb and since they have the ability to infect cells and have their genetic material stably integrated into the host cell with high efficiency. See, e.g., WO 95/30761, WO 95/24929. Oncovirinae require at least one round of target cell proliferation for transfer and integration of exogenous nucleic acid sequences into the patient. Retroviral vectors integrate randomly into the patient's genome. Retroviruses can be used to target stem cells of the nervous system as very few cell divisions take place in other cells of the nervous system (in particular the CNS).

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the W signal, and expressing the gag/pol genes and the env gene from separate transcriptional units. In addition, in some 2, and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

### Expression Vectors

Construction of vectors for recombinant expression of Meteorin polypeptides for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982). Expression vectors may be used for generating producer cells for recombinant production of Meteorin polypeptides for medical use, and for generating therapeutic cells secreting Meteorin polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

For generation of efficient expression vectors, these should contain regulatory sequences necessary for expression of the encoded gene in the correct reading frame. Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, Elongation Factor 1 alpha promoter (EF1-alpha), RSV, CAG. Examples of inducible/repressible promoters include: Tet-On, Tet-Off, Rapamycin-inducible promoter, Mx1, Mo-MLV-LTR, progesterone, RU486.

A group of preferred promoters include CAG, CMV, human UbiC, JeT, SV40, RSV, Tet-regulatable promoter, Mo-MLV-LTR, Mx1, Mt1 and EF-1alpha.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Woodchuck hepatitis virus post-transcriptional regulation element, WPRE, SP163, CMV enhancer, and Chicken [beta]-globin insulator or other insulators.

In one aspect, the invention relates to a vector for use in treatment or prevention of nociceptive pain in a subject.

In one embodiment, the vector for use in treatment and/or prevention of nociceptive pain in a subject comprises a polynucleotide coding for a polypeptide as herein defined.

In another embodiment, the vector further comprises a promoter operably linked to the nucleic acid molecule.

In one embodiment, the vector for use in treatment and/or prevention of nociceptive pain is selected form the group consisting of alphavirus, adenovirus, adeno associated virus, baculovirus, HSV, coronavirus, Bovine papilloma virus, and Mo-MLV, preferably adeno associated virus.

### Cell Lines

In one aspect the invention relates to isolated host cells genetically modified with the vector according to the invention.

The invention also relates to cells suitable for biodelivery of Meteorin via naked or encapsulated cells, which are genetically modified to overexpress Meteorin, and which can be transplanted to the patient to deliver bioactive Meteorin polypeptide locally. Such cells may broadly be referred to as therapeutic cells.

For ex vivo gene therapy, the preferred group of cells includes neuronal cells, neuronal precursor cells, neuronal progenitor cells, neuronal stem cells, human glial stem cells, human precursor cells, stem cells and foetal cells.

For encapsulation the preferred cells include retinal pigmented epithelial cells, including ARPE-19 cells; human immortalised fibroblasts; and human immortalised astrocytes.

The ARPE-19 cell line is a superior platform cell line for encapsulated cell based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic. Methods for culture and encapsulation of ARPE-19 cells are described in U.S. Pat. No. 6,361,771.

In another embodiment the therapeutic cell line is selected from the group consisting of: human fibroblast cell lines, human astrocyte cell lines, human mesencephalic cell line, and human endothelial cell line, preferably immortalised with TERT, SV40T or vmyc.

### Extracellular Matrix

The present invention further comprises culturing Meteorin producing cells in vitro on a extracellular matrix prior to implantation into the mammalian nervous system. The pre-adhesion of cells to microcarriers prior to implantation is designed to enhance the long-term viability of the transplanted cells and provide long term functional benefit.

Materials of which the extracellular matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances, or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of RPE cells. Thus, for example, the matrix material may be extracellular matrix or basement membrane material, which is produced and secreted by RPE cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, see, for example, isher Biotech Source 87-88, Fisher Scientific Co., 1987, pp. 72-75; Sigma Cell Culture Catalog, Sigma Chemical Co., St, Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### EXAMPLES

### Example 1: Repeated Treatment with rmMeteorin Completely Reverses Mechanical Pain in Mice with CFA Inflammatory Hyperalgesia

### Materials and Methods

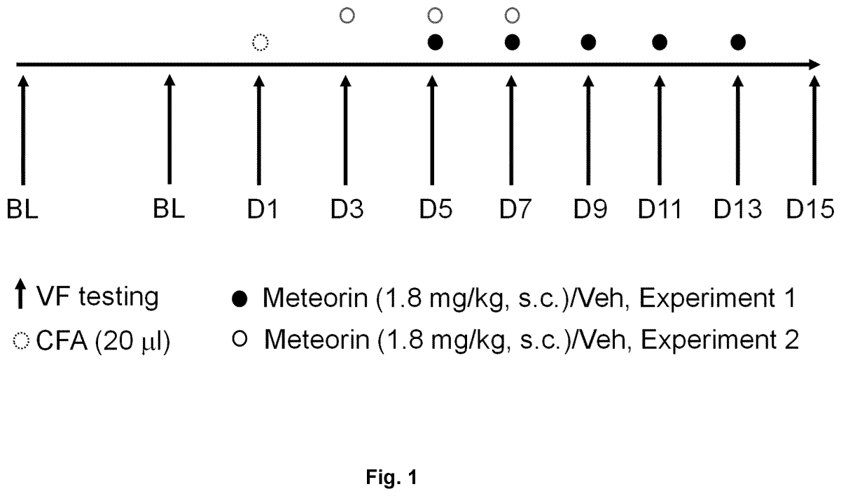

Adult female C57BL/6JRj mice were divided into three groups; (i) complete Freunds adjuvant (CFA)+Vehicle (Dulbecco's PBS), (ii) CFA+rmMeteorin (1.8 mg/kg), (iii) naïve satellites for exposure analysis. Subcutaneous (s.c) injection of either Vehicle or rmMeteorin (solid arrows) was administered every other day (D1, D3, D5, D7, and D9) using an insulin syringe (30G) as shown in FIG. 1. CFA (20 μl; dashed arrow) was injected s.c. under isoflurane anaesthesia (4%, O2 4 ml/min for induction) followed by 2%, 2 ml/min O2 for maintenance). All mice recovered rapidly and were typically active within 5-10 mins upon removal from anaesthesia. No post-operative analgesia was provided to help facilitate full development of CFA-induced sensitization. For behavioural testing, CFA mice were habituated for 15-30 min to clear acrylic behavioural chambers before beginning the experiment. The paw withdrawal threshold (PWT) was tested at baseline and then routinely thereafter using calibrated von Frey filaments until Days 14-15 as a surrogate marker of mechanical, nociceptive hyperalgesia. Paw thickness was measured from the ventral to dorsal aspects across the thickest part of the paw using a digital micrometer before injection of CFA and then routinely afterwards as an index of inflammatory oedema/load. Body weights were measured routinely throughout the duration of the study as a surrogate marker of general welfare. All testing was performed with the experimenter blinded to treatments. Statistical analysis between groups was made using mixed-effects ANOVA. All data are represented as mean±SEM with P<0.05 considered significant.

### Results

At Day 5 all CFA-injected mice developed robust hindpaw mechanical, nociceptive hyperalgesia as shown in FIG. 2a. Four days after the first injection of 1.8 mg/kg rmMeteorin (solid, black circles) an increase in the PWT was observed at Day 9 (P<0.01). With the continued intermittent administration of rmMeteorin the increase in the PWT was maintained at Days 11, 13 and 15 respectively. FIG. 2b clearly shows that CFA injection produced an almost 2 fold increase in paw width indicating the presence of a massive inflammatory oedma which was completely unaffected by rmMeteorin treatment. Finally, body weights of CFA mice treated with rmMeteorin were nearly identical to CFA mice treated with Vehicle (FIG. 2c), suggesting that the general welfare of the mice was not affected by the treatment.

### Conclusions

Treatment with repeated s.c. injections of rmMeteorin completely reversed CFA-induced inflammatory hyperalgesia within days after initiation of dosing. This reversal was maintained throughout the duration of the experiment indicating that analgesic tolerance does not occur with rmMeteorin. rmMeteorin had no effect on inflammatory load indicating that it does not possess a direct anti-inflammatory mechanisms per se, whilst a lack of effect on body weight indicates that the general welfare of rmMeteorin mice was maintained.

### Example 2: Acute Treatment with rmMeteorin Reverses Mechanical Pain Similarly to Repeated Treatment in Mice with CFA Inflammatory Hyperalgesia

### Materials and Methods

Adult female C57BL/6JRj mice were divided into four groups; (i) CFA+Vehicle (ii) CFA+1 injection of Meteorin (iii) CFA+2 injections of rmMeteorin (iv) CFA+3 injections of rmMeteorin. Subcutaneous (s.c) injection of either Vehicle or rmMeteorin was administered every other day (D3, D5 and D9; solid arrows) using an insulin syringe (30G). CFA (20 μl) was injected s.c. under isoflurane anaesthesia (4%, O2 4 ml/min for induction) followed by 2%, 2 ml/min O2 for maintenance). All mice recovered rapidly and were typically active within 5-10 mins upon removal from anaesthesia. No post-operative analgesia was provided to help facilitate full development of CFA-induced sensitization. For behavioural testing, CFA mice were habituated for 15-30 min to clear acrylic behavioural chambers before beginning the experiment. The paw withdrawal threshold (PWT) was tested at baseline and then routinely thereafter using calibrated von Frey filaments until Days 14-15 as a surrogate marker of mechanical, nociceptive hyperalgesia. All testing was performed with the experimenter blinded to treatments. Statistical analysis between groups was made using mixed-effects ANOVA. All data are represented as mean±SEM with P<0.05 considered significant.

### Results

At Day 3 all mice developed robust hindpaw mechanical, nociceptive hyperalgesia induced by CFA injection treatment as shown in FIG. 3a. Four days after a first injection of 1.8 mg/kg rmMeteorin (solid black circles) an increase in the PWT was observed at Day 7 (P<0.01). A similar increase in the PWT at Day 9 was also observed in CFA mice that had received either 2 or 3 injections of rmMeteorin. With the continued intermittent administration, the increase in the PWT was maintained at Days 11, 13 and 14 respectively. At the end of the experiment on Days 14-15, the partial μ-opioid receptor agonist buprenorphine (0.1 mg/kg, s.c.) or Vehicle was administered to mice previously treated with repeated injections of Vehicle to confirm that the sensitivity of the assay conditions used. As expected, buprenorphine produced a full reversal of the PWT (P<0.0001).

### Conclusions

Systemic injection of rmMeteorin fully reversed hindpaw mechanical pain in female mice with CFA-induced inflammatory hyperalgesia. A single injection of rmMeteorin produced a similar magnitude and duration of reversal of nociceptive hyperalgesia as that observed with three injections.

### SEQUENCE OVERVIEW

SEQ ID NO: 1: human Meteorin cDNA
SEQ ID NO: 2: human Meteorin full length amino acid sequence
SEQ ID NO: 3: human Meteorin amino acid sequence without signal peptide
SEQ ID NO: 4: mouse Meteorin cDNA
SEQ ID NO: 5: mouse Meteorin full length amino acid sequence
SEQ ID NO: 6: mouse Meteorin amino acid sequence without signal peptide
SEQ ID NO: 7: rat Meteorin cDNA
SEQ ID NO: 8: rat Meteorin full length amino acid sequence
SEQ ID NO: 9: rat Meteorin amino acid sequence without signal peptide
SEQ ID NO: 10: human codon optimized DNA sequence
SEQ ID NO: 11: mature Meteorin, consensus sequence

Human Meteorin cDNA (1109 bp; CDS = 118-999)
>gi|34147349|ref|NM_024042.2| Homo sapiens hypothetical protein MGC2601
(MGC2601), mRNA
(SEQ ID NO: 1)
GCTTCGCCGGGGCCGGGCGGCCGGCGCCCCCGGCTGCTCCCGCCGCCGCCCGGACCCGCGCCCCGCCGGG
GCAGCGGTGGTGAGAGCCCCGACTCCCCGGACGCCGCCCGCCGTGCCATGGGGTTCCCGGCCGCGGCGCT
GCTCTGCGCGCTGTGCTGCGGCCTCCTGGCCCCGGCTGCCCGCGCCGGCTACTCCGAGGAGCGCTGCAGC
TGGAGGGGCAGCGGCCTCACCCAGGAGCCCGGCAGCGTGGGGCAGCTGGCCCTGGCCTGTGCGGAGGGCG
CGGTTGAGTGGCTGTACCCGGCTGGGGCGCTGCGCCTGACCCTGGGGGGCCCCGATCCCAGAGCGCGGCC
CGGCATCGCCTGTCTGCGGCCGGTGCGGCCCTTCGCGGGCGCCCAGGTCTTCGCGGAGCGCGCAGGGGGC
GCCCTGGAGCTGCTGCTGGCCGAGGGCCCGGGCCCGGCAGGGGGCCGCTGCGTGCGCTGGGGTCCCCGCG
AGCGCCGGGCCCTCTTCCTGCAGGCCACGCCGCACCAGGACATCAGCCGCCGCGTGGCCGCCTTCCGCTT
TGAGCTGCGCGAGGACGGGCGCCCCGAGCTGCCCCCGCAGGCCCACGGTCTCGGCGTAGACGGTGCCTGC
AGGCCCTGCAGCGACGCTGAGCTGCTCCTGGCCGCATGCACCAGCGACTTCGTAATTCACGGGATCATCC
ATGGGGTCACCCATGACGTGGAGCTGCAGGAGTCTGTCATCACTGTGGTGGCCGCCCGTGTCCTCCGCCA
GACACCGCCGCTGTTCCAGGCGGGGCGATCCGGGGACCAGGGGCTGACCTCCATTCGTACCCCACTGCGC
TGTGGCGTCCACCCGGGCCCAGGCACCTTCCTCTTCATGGGCTGGAGCCGCTTTGGGGAGGCCCGGCTGG
GCTGTGCCCCACGATTCCAGGAGTTCCGCCGTGCCTACGAGGCTGCCCGTGCTGCCCACCTCCACCCCTG
CGAGGTGGCGCTGCACTGAGGGGCTGGGTGCTGGGGAGGGGCTGGTAGGAGGGAGGGTGGGCCCACTGCT
TTGGAGGTGATGGGACTATCAATAAGAACTCTGTTCACGCAAAAAAAAAAAAAAAAAAA
Human Meteorin full length amino acid sequence
>IPI00031531.1 REFSEQ_NP: NP_076947 TREMBL: Q9UJH9
ENSEMBL: ENSP00000219542 Tax_Id = 9606 C380A1.2.1 (Novel protein)
(SEQ ID NO: 2)
MGFPAAALLC ALCCGLLAPA ARAGYSEERC SWRGSGLTQE PGSVGQLALA CAEGAVEWLY
PAGALRLTLG GPDPRARPGI ACLRPVRPFA GAQVFAERAG GALELLLAEG PGPAGGRCVR
WGPRERRALF LQATPHQDIS RRVAAFRFEL REDGRPELPP QAHGLGVDGA CRPCSDAELL
LAACTSDFVI HGIIHGVTHD VELQESVITV VAARVLRQTP PLFQAGRSGD QGLTSIRTPL
RCGVHPGPGT FLFMGWSRFG EARLGCAPRF QEFRRAYEAA RAAHLHPCEV ALH
Human Meteorin, protein without signal peptide
(SEQ ID NO: 3)
GYSEERCSWR GSGLTQEPGS VGQLALACAE GAVEWLYPAG ALRLTLGGPD PRARPGIACL
RPVRPFAGAQ VFAERAGGAL ELLLAEGPGP AGGRCVRWGP RERRALFLQA TPHQDISRRV
AAFRFELRED GRPELPPQAH GLGVDGACRP CSDAELLLAA CTSDFVIHGI IHGVTHDVEL
QESVITVVAA RVLRQTPPLF QAGRSGDQGL TSIRTPLRCG VHPGPGTFLF MGWSRFGEAR
LGCAPRFQEF RRAYEAARAA HLHPCEVALH
Mouse Meteorin cDNA, 1363 bp, CDS 84 . . . 959 (SEQ ID NO: 4)
NM_133719. Mus musculus meteorin. [gi: 56550040]
(SEQ ID NO: 4)
gggcagccgc gccgcgggct gctcgcgctg cggccccgac cctcccgggg cagcagtccg
aggccccggc gcgtccccta accatgctgg tagccacgct tctttgcgcg ctctgttgcg
gcctcctggc cgcgtccgct cacgctggct actcggaaga ccgctgcagc tggaggggca
gcggtttgac ccaggagcct ggcagcgtgg ggcagctgac cctggactgt actgagggcg
ctatcgagtg gctgtaccca gctggggcgc tgcgcctgac cctgggcggc cccgatccgg
gcacacggcc cagcatcgtc tgtctgcgcc cagagcggcc cttcgctggt gcccaggtct
tcgctgaacg tatgaccggc aatctagagt tgctactggc cgagggcccg gacctggctg
ggggccgctg catgcgctgg ggtccccgcg agcgccgagc ccttttcctg caggccacac
cacaccgcga catcagccgc agagttgctg ccttccgttt tgaactgcac gaggaccaac
gtgcagaaat gtctccccag gctcaaggtc ttggtgtgga tggtgcctgc aggccctgca
gtgatgccga gctcctcctg gctgcatgca ccagtgattt tgtgatccac gggaccatcc
atggggtcgc ccatgacaca gagctgcaag aatcagtcat cactgtggtg gttgctcgtg
tcatccgcca gacactgcca ctgttcaagg aagggagctc ggagggccaa ggccgggcct
ccattcgtac cttgctgcgc tgtggtgtgc gtcctggccc aggctccttc ctcttcatgg
gctggagccg atttggcgaa gcttggctgg gctgtgctcc ccgcttccaa gagttcagcc
gtgtctattc agctgctctc acgacccatc tcaacccatg tgagatggca ctggactgag
agacctggga gcaagccctg gatggacctt cttctggaga tggggtgttg gggagggtga
tgggagggtg ggtgagaagg gtgtggctcg gatggcatcc tggtacccac agtgagctgg
tagaatacta agtaatctgg accataccag ccactgtagt catggtcttc tgtggcaggc
agcataccca gctctgtgcc tgcctcactt tgtctactct ccagtctgct gcccttctaa
cccttcttag cctgctgacc agtgagctca tgttttcctc gaattccagg gtgctgctgg
ggttcagagc aaccgtgccg tagtttggaa gacttgagct aattgttttt tttttgtttg
tttttttgtt tgtttaaagg tggcctgggg ggggcggcaa aca
Mouse Meteorin full length amino acid sequence
ref|NP_598480.1|meteorin [Mus musculus]
(SEQ ID NO: 5)
MLVATLLCAL CCGLLAASAH AGYSEDRCSW RGSGLTQEPG SVGQLTLDCT EGAIEWLYPA
GALRLTLGGP DPGTRPSIVC LRPERPFAGA QVFAERMTGN LELLLAEGPD LAGGRCMRWG
PRERRALFLQ ATPHRDISRR VAAFRFELHE DQRAEMSPQA QGLGVDGACR PCSDAELLLA
ACTSDFVIHG TIHGVAHDTE LQESVITVVV ARVIRQTLPL FKEGSSEGQG RASIRTLLRC
GVRPGPGSFL FMGWSRFGEA WLGCAPRFQE FSRVYSAALT THLNPCEMAL D
Mouse Meteorin protein without signal peptide
(SEQ ID NO: 6)
GYSEDRCSWR GSGLTQEPGS VGQLTLDCTE GAIEWLYPAG ALRLTLGGPD PGTRPSIVCL RPERPFAGAQ
VFAERMTGNL ELLLAEGPDL AGGRCMRWGP RERRALFLQA TPHRDISRRV AAFRFELHED QRAEMSPQAQ
GLGVDGACRP CSDAELLLAA CTSDFVIHGT IHGVAHDTEL QESVITVVVA RVIRQTLPLF KEGSSEGQGR
ASIRTLLRCG VRPGPGSFLF MGWSRFGEWA LGCAPRGQEF SRVYSAALTT HLNPCEMALD
Rat Meteorin cDNA (1026 bp; CDS = 1-876)
>gi|34870570|ref|XM_213261.2|Rattus norvegicus similar to 1810034B16Rik protein
(LOC287151), mRNA
(SEQ ID NO: 7)
ATGCTGGTAGCGGCGCTTCTCTGCGCGCTGTGCTGCGGCCTCTTGGCTGCGTCCGCTCGAGCTGGCTACT
CCGAGGACCGCTGCAGCTGGAGGGGCAGCGGTTTGACCCAGGAACCTGGCAGCGTGGGGCAGCTGACCCT
GGATTGTACTGAGGGTGCTATCGAGTGGCTGTATCCAGCTGGGGCGCTGCGCCTGACTCTAGGCGGCTCT
GATCCGGGCACGCGGCCCAGCATCGTCTGTCTGCGCCCAACACGGCCCTTCGCTGGTGCCCAGGTCTTCG
CTGAACGGATGGCCGGCAACCTAGAGTTGCTACTGGCCGAGGGCCAAGGCCTGGCTGGGGGCCGCTGCAT
GCGCTGGGGTCCTCGCGAGCGCCGAGCCCTTTTCCTGCAGGCCACGCCACACCGGGACATCAGCCGCAGA
GTTGCTGCCTTCCAATTTGAACTGCACGAGGACCAACGTGCAGAAATGTCTCCCCAGGCCCAAGGTTTTG
GTGTGGATGGTGCCTGCAGGCCCTGCAGTGATGCCGAGCTCCTTCTGACTGCATGCACCAGTGACTTTGT
GATCCATGGGACCATCCATGGGGTCGTCCATGACATGGAGCTGCAAGAATCAGTCATCACTGTGGTGGCC
ACTCGTGTCATCCGCCAGACACTGCCACTGTTCCAGGAAGGGAGCTCGGAGGGCCGGGGCCAGGCCTCCG
TTCGTACCTTGTTGCGCTGTGGTGTGCGTCCTGGCCCAGGCTCCTTCCTCTTCATGGGCTGGAGCCGATT
TGGCGAAGCTTGGCTGGGCTGCGCTCCCCGCTTCCAAGAGTTCAGCCGTGTCTATTCAGCTGCTCTCGCG
GCCCACCTCAACCCATGTGAGGTGGCACTGGACTGAGAGACCTGGGAGCAAGCCCTGGATGGATCTTCCT
CTGGGGATGGGGTGTTGGGGAGGGGTGATAGGAGGGTGGGTGGGAAGGGTGTGGCTCAGATGGCATCCTG
GTACCCACAGTGAGGTGGTAGAATACTAAATAACCTGGATCACACC
Rat Meteorin full length amino acid sequence
>IPI00369281.1|REFSEQ_XP: XP_213621|ENSEMBL: ENSRNOP00000026676
(SEQ ID NO: 8)
MLVAALLCAL CCGLLAASAR AGYSEDRCSW RGSGLTQEPG SVGQLTLDCT EGAIEWLYPA
GALRLTLGGS DPGTRPSIVS LRPTRPFAGA QVFAERMAGN LELLLAEGQG LAGGRCMRWG
PRERRALFLQ ATPHRDISRR VAAFQFELHE DQRAEMSPQA QGFGVDGACR PCSDAELLLT
ACTSDFVIHG TIHGVVHDME LQESVITVVA TRVIRQTLPL FQEGSSEGRG QASVRTLLRC
GVRPGPGSFL FMGWSRFGEA WLGCAPRFQE FSRVYSAALA AHLNPCEVAL D
Rat Meteorin, protein without signal peptide
(SEQ ID NO: 9)
GYSEDRCSWR GSGLTQEPGS VGQLTLDCTE GAIEWLYPAG ALRLTLGGSD PGTRPSIVCL
RPTRPFAGAQ VFAERMAGNL ELLLAEGQGL AGGRCMRWGP RERRALFLQA TPHRDISRRV
AAFQFELHED QRAEMSPQAQ GFGVDGACRP CSDAELLLTA CTSDFVIHGT IHGVVHDMEL
QESVITVVAT RVIRQTLPLF QEGSSEGRGQ ASVRTLLRCG VRPGPGSFLF MGWSRFGEAW
LGCAPRFQEF SRVYSAALAA HLNPCEVALD
Codon optimized Meteorin nucleotide sequence present in constructs pCAn.Meteorin
and pT2.CAn.Meteorin
(SEQ ID NO: 10)
ATGGGCTTTCCCGCTGCCGCCCTGCTGTGCGCTCTGTGCTGCGGACTGCT
GGCTCCTGCAGCCAGAGCCGGCTACAGCGAGGAACGGTGCAGCTGGCGGG
GCAGCGGCCTGACCCAGGAACCTGGCAGCGTCGGCCAGCTCGCACTGGCC
TGTGCAGAAGGCGCCGTGGAGTGGCTGTACCCCGCAGGCGCCCTGAGACT
GACCCTGGGCGGACCCGACCCCAGAGCCAGACCCGGCATTGCCTGTCTGA
GGCCCGTGCGGCCTTTCGCTGGCGCCCAGGTGTTCGCCGAGAGAGCCGGC
GGAGCCCTGGAACTCCTGCTCGCCGAAGGCCCTGGTCCAGCCGGCGGAAG
ATGCGTGAGATGGGGCCCAAGAGAGCGGAGAGCCCTGTTCCTGCAAGCCA
CCCCCCACCAGGACATCAGCAGACGGGTGGCCGCCTTCAGATTCGAGCTG
CGGGAGGACGGTAGACCCGAGCTGCCACCTCAGGCCCACGGACTGGGAGT
GGACGGCGCCTGCAGACCCTGTAGCGACGCCGAGCTGCTGCTCGCCGCCT
GCACCAGCGACTTCGTGATCCACGGCATCATCCACGGCGTGACCCACGAC
GTGGAGCTGCAGGAAAGCGTCATCACCGTCGTCGCCGCCAGAGTGCTGAG
ACAGACCCCCCCTCTGTTCCAGGCCGGCAGAAGCGGCGACCAGGGCCTGA
CCAGCATCCGGACCCCCCTGAGATGCGGCGTGCATCCCGGACCCGGCACC
TTCCTGTTCATGGGCTGGTCCAGATTCGGCGAGGCCCGGCTGGGCTGCGC
TCCCCGGTTCCAGGAATTCAGACGGGCCTACGAGGCCGCCAGGGCCGCTC
ATCTGCACCCCTGCGAGGTGGCCCTGCATTGA
Consensus sequence, mature Meteorin
(SEQ ID NO: 11)
GYSEXRCSWR GSGLTQEPGS VGQLXLXCXE GAXEWLYPAG ALRLTLGGXD PXXRPXIXCL 60
RPXRPFAGAQ VFAERXXGXL ELLLAEGXXX AGGRCXRWGP RERRALFLQA TPHXDISRRV 120
AAFXFELXED XRXEXXPQAX GXGVDGACRP CSDAELLLXA CTSDFVIHGX IHGVXHDXEL 180
QESVITVVXX RVXRQTXPLF XXGXSXXXGX XSXRTXLRCG VXPGPGXFLF MGWSRFGEAX 240
LGCAPRFQEF XRXYXAAXXX HLXPCEXALX 270
X is any of the 21 amino acids that can be encoded by DNA.

### REFERENCES

Armelor, Proc. Natl. Acad. Sci. USA 70: 2702 (1973).
Attal et al. 2010, “EFNS guidelines on the pharmacological treatment of neuropathic pain: 2010 revision”, Eur J Neurol. 2010 September; 17(9):1113-e88. doi: 10.1111/j.1468-1331.2010.02999.x. Epub 2010 Apr. 9.
Fisher Biotech Source 87-88, Fisher Scientific Co., 1987, pp. 72-75.
Banerji et al., Cell 27: 299 (1981).
Benoist and Chambon, Nature 290: 304 (1981).
Breathnach and Chambon, Ann. Rev. Biochem. 50: 349 (1981)).
Capecchi et al., In: Enhancer and eukaryotic gene expression, Gulzman and Shenk, eds., pp. 101-102, Cold Spring Harbor Laboratories (NY 1991).
Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462 Corden et al., Science 209: 1406 (1980).
EMA/CHMP/970057/2011, “Guideline on the clinical development of medicinal products intended for the treatment of pain (EMA/CHMP/970057/2011)” from EMA. FDA 34355740dft.docx 02/07/22, “Draft Development of Non-Opioid Analgesics for Acute Pain Guidance for Industry” from FDA.
Finnerup et al. 2015, “Pharmacotherapy for neuropathic pain in adults: systematic review, meta-analysis and updated NeuPSIG recommendations”, Lancet Neurol. 2015 February; 14(2): 162-173.
Fromm and Berg, J. Mol. Appl. Genetics, 1: 457 (1982).
Gruss et al., Proc. Natl. Acad. Sci. USA 78: 943 (1981).
Hora et al., Bio/Technology, 8:755-758 (1990).
I. E. Creighton, Proteins-Structure and Molecular Properties, 2nd Ed., W. H. Freeman and Company, New York, 1993.
Johnson et al., Nat. Med., 2:795-799 (1996).
Jolly et al., Nucleic Acids Res. 11: 1855 (1983).
Jørgensen et al., Characterization of Meteorin—An evolutionary conserved neurotrophic factor, J mol Neurosci 2009 September; 39 (1-2): 104-116.
Lysaght et al., 56 J. Cell Biochem. 196 (1996).
Maniatis et al., in Molecular Cloning: A Laboratory Manual, Cold Spring Harbor Laboratory, (NY 1982).
Maxam, et al., (Methods in Enzymology, 65: 499, 1980). Messing, et al., (Nucleic Acids Res., 9: 309-, 1981).
Moulin et al. 2014, “Pharmacological management of chronic neuropathic pain: Revised consensus statement from the Canadian Pain Society”, Pain Res Manag. 2014 November-December; 19(6): 328-335.
Moreau et al., Nucleic Acids Res. 9: 6047 (1981).
NICE 2013, “Neuropathic pain in adults: pharmacological management in non-specialist settings”, Clinical guideline [CG173], 20 Nov. 2013.
Nishino et al., “Meteorin: a secret3ed protein that regulates glial cell differentitaion and promotes axonal extension”, EMBO J., 23(9):1998-2008 (2004).
Prockop and Kivirikko, N. Eng. J. Med. 311: 376 (1984).
Rattan et al., Protein Synthesis: Posttranslational Modifications and Aging, Ann. N.Y. Acad. Sci. 663: 48-62, 1992.
Reagan-Shaw et al., FASEB J, 22, 659-661 (2007).
Rossi and deCrombrugghe, Proc. Natl. Acad. Sci. USA 84: 5590-5594 (1987)).
Schmidt et al., Nature 314: 285 (1985).
Seifter et al., Meth. Enzymol. 182: 626-646, 1990.
Sigma Cell Culture Catalog, Sigma Chemical Co., St, Louis, 1991, pp. 162-163
Smith and Niles, Biochem. 19: 1820 (1980).
Ventrex Product Catalog, Ventrex Laboratories, 1989.
Weiss et al., eds., The molecular biology of tumor viruses: RNA tumor viruses, Cold Spring Harbor Laboratory, (NY 1982)).
de Wet et al., J. Biol. Chem., 258: 14385 (1983).
Wold, F., in Posttranslational Covalent Modification of Proteins, B. C. Johnson, Ed.,
Academic Press, New York, pp 1-12, 1983.
Yasuda, Biomed. Ther., 27:1221-1223 (1993).

### Items

1. An isolated polypeptide for use in treatment or prevention of nociceptive pain in a subject, said polypeptide comprising an amino acid sequence selected from the group consisting of:
   i. the amino acid sequence of SEQ ID NO: 3; and
   ii. a biologically active sequence variant of the amino acid sequence of SEQ ID NO:3, wherein the variant has at least 70% sequence identity to SEQ ID NO:3.
2. The polypeptide for the use according to any one of the preceding items, wherein the nociceptive pain is somatic pain or visceral pain.
3. The polypeptide for the use according to any one of the preceding items, wherein the nociceptive pain is inflammatory pain, lower back pain, shoulder pain, musculoskeletal pain, arthritis pain, joint pain, post-operative pain, post-traumatic pain or cancer pain.
4. The polypeptide for the use according to any one of the preceding items, wherein the nociceptive pain is selected from the group consisting of inflammatory pain, and post-operative pain.
5. The polypeptide for the use according to item 3, wherein the nociceptive pain is inflammatory pain, such as inflammatory hyperalgesia.
6. The polypeptide for the use according to item 3, wherein the nociceptive pain is post-operative pain.
7. The polypeptide for use according to item 1, wherein the subject suffers from a disease or disorder selected from the group consisting of arthritis, inflammatory pain, and post-operative pain.
8. The polypeptide for use according to item 6, wherein the arthritis is selected from the group consisting of Osteoarthritis, Rheumatoid arthritis, or Lupus.
9. The polypeptide for use according to item 6, wherein the inflammatory pain is selected from the group consisting of inflammatory hyperalgesia, post-operative pain and arthritis.
10. The polypeptide for the use according to any one of the preceding items, wherein said polypeptide has at least 70% sequence identity to SEQ ID NO: 3, more preferably at least 75%, more preferably at least 80%, more preferably at least 85%, more preferably 90%, more preferably 95%, more preferably 98% sequence identity to SEQ ID NO: 3.
11. The polypeptide for the use according to any one of the preceding items, wherein the polypeptide comprises the consensus sequence of SEQ ID NO:11.
12. The polypeptide for the use according to any one of the preceding items, wherein the polypeptide has cysteine residues at positions 7, 28, 59, 95, 148, 151, 161, 219, 243, and 265 relative to the amino acid sequence of SEQ ID NO:3.
13. The polypeptide for the use according to any one of the preceding items, wherein the polypeptide is a variant polypeptide, wherein any amino acid substitutions are conservative substitutions.
14. The polypeptide for the use according to any one of the preceding items, wherein said polypeptide is capable of forming at least one intramolecular disulfide bridge.
15. The polypeptide for the use according to any one of the preceding items, wherein the subject to be treated is mammalian, preferably primate, more preferably human.
16. The polypeptide for the use according to any one of the preceding items, wherein the polypeptide is administered by systemic administration.
17. The polypeptide for the use according to any one of the preceding items, wherein the polypeptide is administered by parenteral injection, preferably subcutaneous injection or intrathecal injection.
18. The polypeptide for the use according to any one of the preceding items, wherein the polypeptide is administered in dosages of 1 μg/kg-10,000 μg/kg, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body.
19. The polypeptide for the use according to any one of the preceding items, wherein said polypeptide is administered at least 1-3 times weekly, such as 2-5 times weekly, such as 3-6 times weekly.
20. The polypeptide for use according to any one of the preceding items, wherein said polypeptide is administered every other day.
21. The polypeptide for the use according to any one of the preceding items, wherein said polypeptide is administered daily.
22. The polypeptide for the use according to any one of the preceding items, wherein administration of said polypeptide is initiated after onset of symptoms of nociceptive pain.
23. An isolated nucleic acid molecule for use in treatment or prevention of nociceptive pain in a subject, said nucleic acid molecule comprising a nucleic acid sequence coding for a polypeptide comprising an amino acid sequence selected from the group consisting of:
   a. the amino acid sequence of SEQ ID NO: 3;
   b. a biologically active sequence variant of the amino acid sequence of SEQ ID NO:3, wherein the variant has at least 70% sequence identity to SEQ ID NO:3.
24. A vector for use in treatment or prevention of nociceptive pain in a subject, said vector comprising a polynucleotide coding for a polypeptide according to any of the items 1 to 22.
25. The vector for use of item 24, further comprising a promoter operably linked to the nucleic acid molecule.
26. The vector for use of any of the preceding items 24 or 25, wherein the vector is selected form the group consisting of alphavirus, adenovirus, adeno associated virus, baculovirus, HSV, coronavirus, Bovine papilloma virus, and Mo-MLV, preferably adeno associated virus.

1.-15. (canceled)

16. A method for treatment or prevention of nociceptive pain in a subject in need thereof, the method comprising administering a therapeutically effective amount of an isolated polypeptide comprising:
i. the amino acid sequence of SEQ ID NO: 3; or
ii. a biologically active sequence variant of the amino acid sequence of SEQ ID NO:3, wherein the variant has at least 85% sequence identity to SEQ ID NO:3,
to the subject in need thereof.

17. The method according to claim 16, wherein the nociceptive pain arises from actual or threatened damage to non-neural tissue and is due to the activation of nociceptors.

18. The method according to claim 16, wherein the nociceptive pain is somatic pain or visceral pain.

19. The method according to claim 16, wherein the nociceptive pain is inflammatory pain, lower back pain, shoulder pain, musculoskeletal pain, arthritis pain, joint pain, post-operative pain, post-traumatic pain or cancer pain.

20. The method according to claim 16, wherein the nociceptive pain is inflammatory pain, or post-operative pain.

21. The method according to claim 19, wherein the nociceptive pain is inflammatory pain.

22. The method according to claim 16, wherein the subject suffers from arthritis.

23. The method according to claim 22, wherein the arthritis is Osteoarthritis, Rheumatoid arthritis, or Lupus.

24. The method according to claim 16, wherein said polypeptide has at least 90% sequence identity to SEQ ID NO: 3.

25. The method according to claim 16, wherein said polypeptide has at least 95% sequence identity to SEQ ID NO: 3.

26. The method according to claim 16, wherein said polypeptide has at least 98% sequence identity to SEQ ID NO: 3.

27. The method according to claim 16, wherein the polypeptide comprises the consensus sequence of SEQ ID NO:11.

28. The method according to claim 16, wherein the polypeptide is administered by systemic administration.

29. The method according to claim 16, wherein the polypeptide is administered by parenteral injection.

30. The method according to claim 16, wherein the polypeptide is administered by intravenous injection, subcutaneous injection or intrathecal injection.

31. The method according to claim 16, wherein the polypeptide is administered in dosages of 1 μg/kg-10,000 μg/kg, 1 μg/kg-7,500 μg/kg, 1 μg/kg-5,000 μg/kg, 1 μg/kg-2,000 μg/kg, 1 μg/kg-1,000 μg/kg, 1 μg/kg-700 μg/kg, 5 μg/kg-500 μg/kg, or 10 μg/kg to 100 μg/kg body.

32. The method according to claim 16, wherein said polypeptide is administered at least 1-3 times weekly, 2-5 times weekly, or 3-6 times weekly.

33. The method according to claim 16, wherein the subject to be treated is human.

34. A method for treatment or prevention of nociceptive pain in a subject in need thereof, the method comprising administering a therapeutically effective amount of an isolated nucleic acid molecule comprising a nucleic acid sequence coding for a polypeptide comprising:
a. the amino acid sequence of SEQ ID NO: 3; or
b. a biologically active sequence variant of the amino acid sequence of SEQ ID NO:3, wherein the variant has at least 85% sequence identity to SEQ ID NO:3
to the subject in need thereof.

35. A method for treatment or prevention of nociceptive pain in a subject in need thereof, the method comprising administering a therapeutically effective amount of a vector comprising a polynucleotide coding for a polypeptide according to claim 1 to the subject in need thereof.
